(19) United States
(12) Patent Application Publication
(10) Pub. No.: US 2014/0371299 A1
(43) Pub. Date: Dec. 18, 2014

(54) Use of Apoptosis-Specific elF-5A siRNA to Down Regulate Expression of Proinflammatory Cytokines to Treat Sepsis

(71) Applicant: Senesco Technologies, Inc., New Brunswick, NJ (US)

(72) Inventors: JOHN E. THOMPSON, WATERLOO (CA); CATHERINE TAYLOR, WATERLOO (CA); CHARLES A. DINARELLO, DENVER, CO (US)

(73) Assignee: Senesco Technologies, Inc., New Brunswick, NJ (US)

(21) Appl. No.: 14/475,389

(22) Filed: Sep. 2, 2014

Related U.S. Application Data
(63) Continuation-in-part of application No. 13/487,951, filed on Jun. 4, 2012, now abandoned.
Application No. 13/487,951 is a division of application No. 11/725,520, filed on Mar. 20, 2007, now Pat. No. 8,193,158.
Application No. 11/725,520 is a continuation-in-part of application No. 14/264,517, filed on Apr. 29, 2014.
Application No. 14/264,517 is a division of application No. 13/099,171, filed on May 2, 2011, now Pat. No. 8,754,057.
Application No. 13/099,171 is a continuation of application No. 11/134,445, filed on May 23, 2005, now abandoned.
Application No. 11/134,445 is a continuation of application No. 10/861,980, filed on Jun. 7, 2004, now abandoned.
(60) Provisional application No. 60/783,413, filed on Mar. 20, 2006.
(60) Provisional application No. 60/798,333, filed on May 8, 2006.

(51) Int. Cl. C12N 15/113

(52) U.S. Cl. CPC C12N 15/113; C12N 2310/11
USPC 514/44 A

(57) ABSTRACT

The present invention relates to apoptosis specific eucaryotic initiation factor 5A (eIF-5A), referred to as apoptosis-specific eIF-5A or eIF5-A1, nucleic acids and polypeptides and methods for down regulating pro-inflammatory cytokines in a mammal by administering siRNA against eIF-5A1 to the mammal to treat/prevent sepsis and/or hemorrhagic shock.

### SEQUENCE LISTING SUBMISSION VIA EFS-WEB

A computer readable text file, entitled “061945-5001-02-SequenceListing.txt” created on or about Aug. 1, 2014, with a file size of about 51 kb contains the sequence listing for this application and is hereby incorporated by reference in its entirety.

### FIELD OF THE INVENTION

The present invention relates to apoptosis-specific eucaryotic initiation factor (“eIF-5A”) or referred to as “apoptosis-specific eIF-5A” or “eIF-5A1.”

### BACKGROUND OF THE INVENTION

Apoptosis is a genetically programmed cellular event that is characterized by well-defined morphological features, such as cell shrinkage, chromatin condensation, nuclear fragmentation, and membrane blebbing. Kerr et al. (1972) Br. J. Cancer, 26, 239-257; Wyllie et al. (1980) Int. Rev. Cytol., 68, 251-306. It plays an important role in normal tissue development and homeostasis, and defects in the apoptotic program are thought to contribute to a wide range of human disorders ranging from neurodegenerative and autoimmunity disorders to neoplasms. Thompson (1995) Science, 267, 1456-1462; Mullauer et al. (2001) Mutat. Res, 488, 211-231. Although the morphological characteristics of apoptotic cells are well characterized, the molecular pathways that regulate this process have only begun to be elucidated.

One group of proteins that is thought to play a key role in apoptosis is a family of cysteine proteases, termed caspases, which appear to be required for most pathways of apoptosis. Creagh & Martin (2001) Biochem. Soc. Trans, 29, 696-701; Dales et al. (2001) Leuk. Lymphoma, 41, 247-253. Caspases trigger apoptosis in response to apoptotic stimuli by cleaving various cellular proteins, which results in classic manifestations of apoptosis, including cell shrinkage, membrane blebbing and DNA fragmentation. Chang & Yang (2000) Microbiol. Mol. Rev., 64, 821-846.

Pro-apoptotic proteins, such as Bax or Bak, also play a key role in the apoptotic pathway by releasing caspase-activating molecules, such as mitochondrial cytochrome c, thereby promoting cell death through apoptosis. Martinou & Green (2001) Nat. Rev. Mol. Cell. Biol., 2, 63-67; Zou et al. (1997) Cell, 90, 405-413. Anti-apoptotic proteins, such as Bcl-2, promote cell survival by antagonizing the activity of the pro-apoptotic proteins, Bax and Bak. Tsujimoto (1998) Genes Cells, 3, 697-707; Kroemer (1997) Nature Med., 3, 614-620. The ratio of Bax:Bcl-2 is thought to be one way in which cell fate is determined; an excess of Bax promotes apoptosis and an excess of Bcl-2 promotes cell survival. Salomons et al. (1997) Int. J. Cancer, 71, 959-965; Wallace-Brodeur & Lowe (1999) Cell Mol. Life Sci., 55, 64-75.

Another key protein involved in apoptosis is a protein that encoded by the tumor suppressor gene p53. This protein is a transcription factor that regulates cell growth and induces apoptosis in cells that are damaged and genetically unstable, presumably through up-regulation of Bax. Bold et al. (1997) Surgical Oncology, 6, 133-142; Ronen et al., 1996; Schuler & Green (2001) Biochem. Soc. Trans., 29, 684-688; Ryan et al. (2001) Curr. Opin. Cell Biol., 13, 332-337; Zörnig et al. (2001) Biochem. Biophys. Acta, 1551, F1-F37.

Alterations in the apoptotic pathways are believed to play a key role in a number of disease processes, including cancer. Wyllie et al. (1980) Int. Rev. Cytol., 68, 251-306; Thompson (1995) Science, 267, 1456-1462; Sen & D'Incalci (1992) FEBS Letters, 307, 122-127; McDonnell et al. (1995) Seminars in Cancer and Biology, 6, 53-60. Investigations into cancer development and progression have traditionally been focused on cellular proliferation. However, the important role that apoptosis plays in tumorigenesis has recently become apparent. In fact, much of what is now known about apoptosis has been learned using tumor models, since the control of apoptosis is invariably altered in some way in tumor cells. Bold et al. (1997) Surgical Oncology, 6, 133-142.

Cytokines also have been implicated in the apoptotic pathway. Biological systems require cellular interactions for their regulation, and cross-talk between cells generally involves a large variety of cytokines. Cytokines are mediators that are produced in response to a wide variety of stimuli by many different cell types. Cytokines are pleiotropic molecules that can exert many different effects on many different cell types, but are especially important in regulation of the immune response and hematopoietic cell proliferation and differentiation. The actions of cytokines on target cells can promote cell survival, proliferation, activation, differentiation, or apoptosis depending on the particular cytokine, relative concentration, and presence of other mediators.

The use of anti-cytokines to treat autoimmune disorders such as psoriasis, rheumatoid arthritis, and Crohn's disease is gaining popularity. The pro-inflammatory cytokines IL-1 and TNF play a large role in the pathology of these chronic disorders. Anti-cytokine therapies that reduce the biological activities of these two cytokines can provide therapeutic benefits (Dinarello and Abraham, 2002).

Interleukin 1 (IL-I) is an important cytokine that mediates local and systemic inflammatory reactions and which can synergize with TNF in the pathogenesis of many disorders, including vasculitis, osteoporosis, neurodegenerative disorders, diabetes, lupus nephritis, and autoimmune disorders such as rheumatoid arthritis. The importance of IL-1β in tumour angiogenesis and invasiveness was also recently demonstrated by the resistance of IL-1β knockout mice to metastases and angiogenesis when injected with melanoma cells (Voronov et al., 2003).

Interleukin 18 (IL-18) is a recently discovered member of the IL-1 family and is related by structure, receptors, and function to IL-1. IL-18 is a central cytokine involved in inflammatory and autoimmune disorders as a result of its ability to induce interferon-gamma (IFN-γ), TNF-α, and IL-1. IL-1β and IL-18 are both capable of inducing production of TNF-α, a cytokine known to contribute to cardiac dysfunction during myocardial ischemia (Maekawa et al., 2002) Inhibition of IL-18 by neutralization with an IL-18 binding protein was found to reduce ischemia-induced myocardial dysfunction in an ischemia/reperfusion model of suprafused human atrial myocardium (Dinarello, 2001). Neutralization of IL-18 using a mouse IL-18 binding protein was also able to decrease IFN-γ, TNF-α, and IL-1β transcript levels and reduce joint damage in a collagen-induced arthritis mouse model (Banda et al., 2003). A reduction of IL-18 production or availability may also prove beneficial to control metastatic cancer as injection of IL-18 binding protein in a mouse melanoma model successfully inhibited metastases (Carrascal et al., 2003). As a further indication of its importance as a pro-inflammatory cytokine, plasma levels of IL-18 were elevated in patients with chronic liver disease and increased levels were correlated with the severity of the disease (Ludwiczek et al., 2002). Similarly, IL-18 and TNF-α were elevated in the serum of diabetes mellitus patients with nephropathy (Moriwaki et al., 2003). Neuroinflammation following traumatic brain injury is also mediated by pro-inflammatory cytokines and inhibition of IL-18 by the IL-18 binding protein improved neurological recovery in mice following brain trauma (Yatsiv et al., 2002).

TNF-α, a member of the TNF family of cytokines, is a pro-inflammatory cytokine with pleiotropic effects ranging from co-mitogenic effects on hematopoietic cells, induction of inflammatory responses, and induction of cell death in many cell types. TNF-α is normally induced by bacterial lipopolysaccharides, parasites, viruses, malignant cells and cytokines and usually acts beneficially to protect cells from infection and cancer. However, inappropriate induction of TNF-α is a major contributor to disorders resulting from acute and chronic inflammation such as autoimmune disorders and can also contribute to cancer, AIDS, heart disease, and sepsis (reviewed by Aggarwal and Natarajan, 1996; Sharma and Anker, 2002). Experimental animal models of disease (i.e. septic shock and rheumatoid arthritis) as well as human disorders (i.e. inflammatory bowel diseases and acute graft-versus-host disease) have demonstrated the beneficial effects of blocking TNF-α (Wallach et al., 1999). Inhibition of TNF-α has also been effective in providing relief to patients suffering autoimmune disorders such as Crohn's disease (van Deventer, 1999) and rheumatoid arthritis (Richard-Miceli and Dougados, 2001). The ability of TNF-α to promote the survival and growth of B lymphocytes is also thought to play a role in the pathogenesis of B-cell chronic lymphocytic leukemia (B-CLL) and the levels of TNF-α being expressed by T cells in B-CLL was positively correlated with tumour mass and stage of the disease (Bojarska-Junak et al., 2002). Interleukin-1β (IL-1β) is a cytokine known to induce TNF-α production.

The amino acid sequence of eIF-5A is well conserved between species, and there is strict conservation of the amino acid sequence surrounding the hypusine residue in eIF-5A, which suggests that this modification may be important for survival. Park et al. (1993) Biofactors, 4, 95-104. This assumption is further supported by the observation that inactivation of both isoforms of eIF-5A found to date in yeast, or inactivation of the DHS gene, which catalyzes the first step in their activation, blocks cell division. Schnier et al. (1991) Mol. Cell. Biol., 11, 3105-3114; Sasaki et al. (1996) FEBS Lett., 384, 151-154; Park et al. (1998) J. Biol. Chem., 273, 1677-1683. However, depletion of eIF-5A protein in yeast resulted in only a small decrease in total protein synthesis suggesting that eIF-5A may be required for the translation of specific subsets of mRNA's rather than for protein global synthesis. Kang et al. (1993), “Effect of initiation factor eIF-5A depletion on cell proliferation and protein synthesis,” in Tuite, M. (ed.), Protein Synthesis and Targeting in Yeast, NATO Series H. The recent finding that ligands that bind eIF-5A share highly conserved motifs also supports the importance of eIF-5A. Xu & Chen (2001) J. Biol. Chem., 276, 2555-2561. In addition, the hypusine residue of modified eIF-5A was found to be essential for sequence-specific binding to RNA, and binding did not provide protection from ribonucleases.

In addition, intracellular depletion of eIF-5A results in a significant accumulation of specific mRNAs in the nucleus, indicating that eIF-5A may be responsible for shuttling specific classes of mRNAs from the nucleus to the cytoplasm. Liu & Tartakoff (1997) Supplement to Molecular Biology of the Cell, 8, 426a. Abstract No. 2476, 37th American Society for Cell Biology Annual Meeting. The accumulation of eIF-5A at nuclear pore-associated intranuclear filaments and its interaction with a general nuclear export receptor further suggest that eIF-5A is a nucleocytoplasmic shuttle protein, rather than a component of polysomes. Rosorius et al. (1999) J. Cell Science, 112, 2369-2380.

The first cDNA for eIF-5A was cloned from human in 1989 by Smit-McBride et al., and since then cDNAs or genes for eIF-5A have been cloned from various eukaryotes including yeast, rat, chick embryo, alfalfa, and tomato. Smit-McBride et al. (1989) J. Biol. Chem., 264, 1578-1583; Schnier et al. (1991) (yeast); Sano, A. (1995) in Imahori, M. et al. (eds), Polyamines, Basic and Clinical Aspects, VNU Science Press, The Netherlands, 81-88 (rat); Rinaudo & Park (1992) FASEB J., 6, A453 (chick embryo); Pay et al. (1991) Plant Mol. Biol., 17, 927-929 (alfalfa); Wang et al. (2001) J. Biol. Chem., 276, 17541-17549 (tomato).

### SUMMARY OF INVENTION

The present invention relates to apoptosis specific eucaryotic initiation factor 5A (eIF-5A), referred to as “apoptosis specific eIF-5A” or “eIF-5A1” and methods for inhibiting or suppressing apoptosis in cells using antisense nucleotides or siRNAs to inhibit expression of apoptosis-specific eIF-5A.

The present invention also relates to methods of increasing apoptosis in cells by increasing expression of apoptosis-specific eIF-5A.

The present invention relates to apoptosis specific eucaryotic initiation factor 5A (eIF-5A), referred to as “apoptosis specific eIF-5A” or “eIF-5A1.” The invention also relates to suppressing or inhibiting expression of pro-inflammatory cytokines in a subject, including a human, in vivo, (and in vitro in a cell) by inhibiting expression of apoptosis-specific eIF-5A through the use of eIF5A1 siRNAs or antisense polynucleotides. eIF5A1 siRNA and antisense constructs of eIF5A1 are administered to decrease expression of pro-inflammatory cytokines such as IL-1β, IL-2, IL-4, IL-5, IL-10, IFN-γ, TNF-α, IL-3, IL-6, IL-12(p40), IL-12(p70), G-CSF, KC, MIP-1a, and RANTES, which is useful in the treatment or prevention of sepsis and/or hemorrhagic induced shock.

The present invention also provides a pharmaceutical composition for decreasing expression of pro-inflammatory cytokines, comprising eIF5A1 siRNA and a pharmaceutically acceptable carrier. Pharmaceutical compositions of the invention may be administered to treat or prevent the onset of sepsis in a subject, including a human. In certain embodiments, the pharmaceutical composition comprises the nucleotide sequence CGG AAU GAC UUC CAG CUG A (SEQ ID NO: 117).

### BRIEF DESCRIPTION OF THE DRAWINGS

FIG. 1 shows the effects of siRNA against eIF-5A1 on the effect of proinflammatory cytokines FIG. 1 shows that siRNA against eIF-5A1 causes decreased expression of IL-1β.

FIG. 2 shows that siRNA against eIF-5A1 causes decreased expression of IL-2.

FIG. 3 shows that siRNA against eIF-5A1 causes decreased expression of IL-4.

FIG. 4 shows that siRNA against eIF-5A1 causes decreased expression of IL-5.

FIG. 5 shows that siRNA against eIF-5A1 causes decreased expression of IL-10.

FIG. 6 shows that siRNA against eIF-5A1 causes increased expression of GM-CSF.

FIG. 7 shows that siRNA against eIF-5A1 causes decreased expression of IFN-γ.

FIG. 8 shows that siRNA against eIF-5A1 causes decreased expression of TNF-α.

FIG. 9 shows that siRNA against eIF-5A1 causes increased expression of IL-1α.

FIG. 10 shows that siRNA against eIF-5A1 causes decreased expression of IL-3.

FIG. 11 shows that siRNA against eIF-5A1 causes decreased expression of IL-6.

FIG. 12 shows that siRNA against eIF-5A1 causes decreased expression of IL-12(p40).

FIG. 13 shows that siRNA against eIF-5A1 causes decreased expression of IL-12(p70).

FIG. 14 shows that siRNA against eIF-5A1 causes increased expression of IL-17.

FIG. 15 shows that siRNA against eIF-5A1 causes decreased expression of G-CSF.

FIG. 16 shows that siRNA against eIF-5A1 causes decreased expression of KC.

FIG. 17 shows that siRNA against eIF-5A1 causes decreased expression of MIP-1α.

FIG. 18 shows that siRNA against eIF-5A1 causes decreased expression of RANTES.

FIG. 19 provides an eIF-5A1 siRNA construct.

FIG. 20 shows the effect of cardiac puncture and bleeding on one hour post hemorrhagic lung. IL-1β expression significantly increases.

FIG. 21 shows that administration of eIF5A1 siRNA prior to inducement of hemorrhage shock, caused a decreased expression of Il-1B and TNF-α.

FIG. 22 provides the nucleotide sequence of human eIF5A1 aligned against eIF5A2.

FIG. 23 provides the amino acid sequence of human eIF5A1 aligned against eIF5A2.

FIG. 24 provides the nucleotide sequence of human eIF5A1 with exemplary antisense oligonucleotides.

FIG. 25 provides the nucleotide sequence of human eIF5A1 with exemplary antisense oligonucleotides.

FIGS. 26A and B provide the nucleotide sequence of human eIF5A1 with exemplary siRNAs.

FIG. 27 provides the nucleotide sequence of human eIF5A1 with exemplary siRNAs.

FIG. 28 shows that intraveneous delivery of siRNAs directed against apoptosis-specific eIF-5A cause a decrease in levels of TNF-α in the serum.

FIG. 29 shows that transnasal delivery of siRNAs directed against apoptosis-specific eIF-5A cause a decrease in levels of TNF-α in the lung.

FIG. 30 shows that transnasal delivery of siRNAs directed against apoptosis-specific eIF-5A cause a decrease in levels of MIP-1 in the lung.

FIG. 31 shows that intranasal delivery of siRNAS directed against apoptosis-specific eIF-5A cause a decrease in the levels of IL-1α.

FIG. 32 shows that after mice received LPS and eIF-5A1 siRNA intranasaly had a reduced myeloperoxidase activity than mice receiving control siRNA.

FIG. 33 shows that nasal-LPS-induced loss of thymocyes is blocked by pre-treatment with apoptosis-specific eIF-5A siRNA.

FIG. 34 shows the time course for experiments with intranasal delivery of apoptosis-specific eIF-5A siRNA.

FIG. 35 shows that nasal-LPS-induced loss of thymocyes is blocked by pre-treatment with apoptosis-specific eIF-5A siRNA.

FIGS. 36A-E show that siRNA against eIF-5A decreased production of IL-6, IFN-γ and Il-1α.

FIG. 37 shows that siRNA against eIF-5A is able to reduce the expression of TNFα as a result of treatment with LPS. The top panel shows the raw data and the bottom panel shows the data in a bar graph.

FIG. 38 shows the results of an experiment where septic Balb/C mice were treated with different concentrations of siRNA and at different times.

FIG. 39 shows the results of FIG. 38 in a different format.

FIG. 40 shows the results of an experiment where septic C57BL/6 mice were treated with different concentrations of siRNA and at different times.

FIG. 41 shows the results of FIG. 40 in a different format.

FIG. 42-44 show the results of a combined sepsis survival study in Balb/C mice. This study shows that mice receiving apoptosis-specific eIF-5A survived longer than control mice.

FIGS. 45-47 show the results of a combined sepsis survival study in C57BL/6 mice. This study shows that mice receiving apoptosis-specific eIF-5A siRNA survived longer than control mice.

FIG. 48 summarized the sepsis study, showing that animals treated with apoptosis-specific eIF-5A siRNA had a better chance of survival.

### DETAILED DESCRIPTION OF THE INVENTION

Several isoforms of eukaryotic initiation factor 5A (“eIF-5A”) have been isolated and present in published databanks. It was thought that these isoforms were functionally redundant. The present inventors have discovered that one isoform is upregulated immediately before the induction of apoptosis, which they have designated apoptosis-specific eIF-5A or eIF-5A1. The subject of the present invention is apoptosis-specific eIF-5A and the down regulation of its expression to down regulate expression of pro-inflammatory cytokines

The present invention provides a method of treating pathological conditions characterized by an increased IL-1, TNF-alpha, IL-61 or IL-18 level comprising administering to a mammal having said pathological condition, agents to reduce expression of apoptosis-specific eIF-5A as described above (antisense oligonucleotides and siRNA).

Known pathological conditions characterized by an increase in IL-1, TNF-alpha, or 11-6 levels include, but are not limited to arthritis-rheumatoid and osteo arthritis, asthma, allergies, arterial inflammation, crohn's disease, inflammatory bowel disease, (ibd), ulcerative colitis, coronary heart disease, cystic fibrosis, diabetes, lupus, multiple sclerosis, graves disease, periodontitis, glaucoma & macular degeneration, ocular surface diseases including keratoconus, organ ischemia-heart, kidney, repurfusion injury, sepsis, multiple myeloma, organ transplant rejection, psoriasis and eczema. Thus, reducing expression of apoptosis-specific eIF-5A with the antisense oligonucleotides, siRNAs and methods of the present invention, may provide relief from these pathological conditions.

Many important human diseases are caused by abnormalities in the control of apoptosis. These abnormalities can result in either a pathological increase in cell number (e.g. cancer) or a damaging loss of cells (e.g. degenerative diseases). As non-limiting examples, the methods and compositions of the present invention can be used to prevent or treat the following apoptosis-associated diseases and disorders: neurological/neurodegenerative disorders (e.g., Alzheimer's, Parkinson's, Huntington's, Amyotrophic Lateral Sclerosis (Lou Gehrig's Disease), autoimmune disorders (e.g., rheumatoid arthritis, systemic lupus erythematosus (SLE), multiple sclerosis), Duchenne Muscular Dystrophy (DMD), motor neuron disorders, ischemia, heart ischemia, chronic heart failure, stroke, infantile spinal muscular atrophy, cardiac arrest, renal failure, atopic dermatitis, sepsis and septic shock; AIDS, hepatitis, glaucoma, diabetes (type 1 and type 2), asthma, retinitis pigmentosa, osteoporosis, xenograft rejection, and burn injury.

Sepsis is a process of malignant intravascular inflammation causing ˜210,000 deaths annually. Accordingly, adjunctive therapies are needed. Sepsis is also known as systemic inflammatory response syndrome (“SIRS”). Sepsis is caused by bacterial infection that can originate anywhere in the body. Sepsis can be simply defined as a spectrum of clinical conditions caused by the immune response of a patient to infection that is characterized by systemic inflammation and coagulation. It includes the full range of response from systemic inflammatory response (SIRS) to organ dysfunction to multiple organ failure and ultimately death.

Sepsis is a very complex sequence of events and much work still needs to be done to completely understand how a patient goes from SIRS to septic shock. Patients with septic shock have a biphasic immunological response. Initially they manifest an overwhelming inflammatory response to the infection. This is most likely due to the pro-inflammatory cytokines Tumor Necrosis Factor (TNF), IL-1, IL-12, Interferon gamma (IFN-γ), and IL-6. The body then regulates this response by producing anti-inflammatory cytokines (IL-10), soluble inhibitors (TNF receptors, IL-1 receptor type II, and IL-1RA (an inactive form of IL-1)), which is manifested in the patient by a period of immunodepression. Persistence of this hypo-responsiveness is associated with increased risk of nosocomial infection and death.

This systemic inflammatory cascade is initiated by various bacterial products. These bacterial products such as gram-negative bacteria=endotoxin, formyl peptides, exotoxins, and proteases; gram-positive bacteria=exotoxins, superantigens (toxic shock syndrome toxin (TSST), streptococcal pyrogenic exotoxin A (SpeA)), enterotoxins, hemolysins, peptidoglycans, and lipotechoic acid, and fungal cell wall material, which bind to cell receptors on the host's macrophages and activate regulatory proteins such as Nuclear Factor Kappa B (NFkB). Endotoxin activates the regulatory proteins by interacting with several receptors. The CD receptors pool the LPS-LPS binding protein complex on the surface of the cell and then the TLR receptors translate the signal into the cells.

As mentioned above, the pro-inflammatory cytokines produced are tumor necrosis factor (TNF), Interleukins 1, 6 and 12 and Interferon gamma (IFN-γ). These cytokines can act directly to affect organ function or they may act indirectly through secondary mediators. The secondary mediators include nitric oxide, thromboxanes, leukotrienes, platelet-activating factor, prostaglandins, and complement. TNF and IL-1 (as well as endotoxin) can also cause the release of tissue-factor by endothelial cells leading to fibrin deposition and disseminated intravascular coagulation (DIC).

These primary and secondary mediators then cause the activation of the coagulation cascade, the complement cascade and the production of prostaglandins and leukotrienes. Clots lodge in the blood vessels which lowers profusion of the organs and can lead to multiple organ system failure. In time, this activation of the coagulation cascade depletes the patient's ability to make a clot resulting in DIC and ARDS.

The cumulative effect of this cascade is an unbalanced state, with inflammation dominant over anti-inflammation and coagulation dominant over fibrinolysis. Microvascular thrombosis, hypoperfusion, ischemia, and tissue injury result. Severe sepsis, shock, and multiple organ dysfunction may occur, leading to death.

Because the present inventors had previously determined that eIF5A1 siRNA (delivered intranasaly as naked siRNA) decreased the production or expression of multiple potential mediators of sepsis (e.g. IL-1(β, TNF-α, IL-8, iNOS, TLR-4 expression) in cell systems and a few proinflammatory cytokines in blood following intranasal lipopolysaccharide (LPS) challenge in vivo, the impact on survival and cytokine expression in endotoxemic mice was studied. See co-pending U.S. application Ser. No. 11/134,445 (filed May 23, 2005), Ser. No. 11/184,982 (filed Jul. 20, 2005), Ser. No. 11/293,391 (filed Nov. 28, 2005), and Ser. No. 11/595,990 (filed Nov. 13, 2006), which are all herein incorporated by reference in their entirety.

BALB/C mice were inoculated with E. coli 0111:B4 LPS intraperitoneally (IP), causing death in 93% of controls. Animals received either eIF5A1 siRNA (N=5) (3′-GCC UUA CUG AAG GUC GAC U -5′) or scrambled RNA as a control (N=15). A 50 μg dose of eIF5A1 siRNA was given IP in conjunction with 100 μg of transfection micelle comprised of DOTAP. The siRNA-liposome complex was dosed at t=−48 and −24 hrs prior to LPS administration. Survival experiments were conducted and under similar conditions mice were sacrificed at 90 min or 8 hours after LPS administration and blood sampled. A bead-based multiplex sandwich immunoassay quantified circulating cytokines. The results indicate that treatment of BALB/C mice with eIF5A1 siRNA conferred 60% protection (p<0.01). With treatment, IL-1β dropped from 5909 to 658 pg/mL at 90 min and from 2478 to 1032 pg/mL at 8 hrs. Treatment also decreased INF-α from 33649 to 3696 pg/mL at 90 min and from 1272 to 901 at 8 hrs. MIP-1α also decreased from 10499 to 3475 pg/mL at 90 min and from 680 to 413 pg/mL at 8 hrs with treatment. At 8 hrs, treatment reduced IFN-γ from 142 to 86 pg/mL and IL-12(p40) from 46570 to 14261 pg/mL. The anti-inflammatory cytokine IL-10 was increased from 719 to 898 pg/mL at 90 min with treatment. These studies show that targeting inflammatory mediators with siRNA confers protection in endotoxemic mice and suggests this may be a useful approach in the treatment of septic patients.

In addition, to the septic model discussed above, the inventors also developed a novel murine model for studying hemorrhagic shock. In this model, male mice C-57BL/6J (8-12 weeks old) were induced into hemorrhage shock by withdrawal of 30% of the calculated blood volume (0.55 ml) by cardiac puncture over a 60-sec period (under methoxyflurane anesthesia). Lungs were harvested at 1 h after bleeding and were homogenized in 1 ml of ice-cold extraction buffer containing 20 mM HEPES (pH 7.4), 20 mM glycerophosphate, 20 mM sodium pyrophosphate, 0.2 mM Na3VO4, 2 mM EDTA, 20 mM sodium fluoride, 10 mM benzamidine, 1 mM DTT, 20 ng/ml leupeptin, 0.4 mM Pefabloc SC, and 0.01% Triton X-100. The homogenate was centrifuged at 14,000 g for 15 min at 4° C. The supernatant was collected, and the protein concentration was determined with the bicinchoninic acid assay. The resulting supernatant was used for determination of TNF, IL-1, and IL-6 by ECL (liquid phase ELISA), according to the manufacturer's suggestions. Final results were expressed as picograms cytokine protein per milligram of protein.

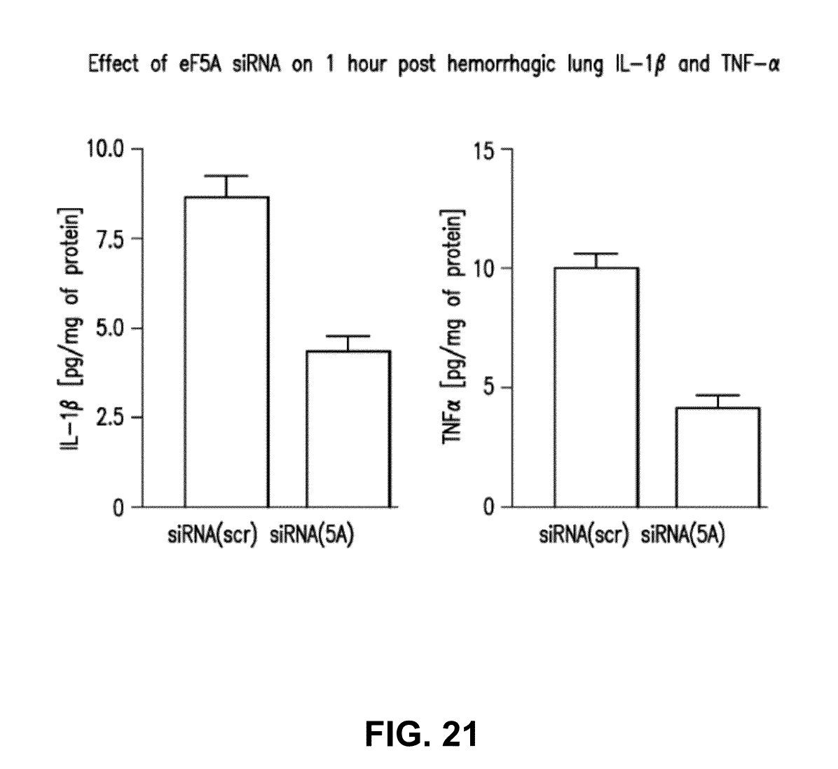

In another hemorrhagic model, the inventors showed that providing eIF5A siRNA, they could reducing expression of TNFα and IL-1β. 5 Mice C-57BL/6J, male induced i.p. were treated with 50 μg of eF5A1 siRNA 24 hours prior to hemorrhage. In the control, 5 Mice C-57BL/6J, male induced i.p. were treated with 50 ug of scrambled siRNA 24 hours prior to hemorrhage. Hemorrhage shock was developed by withdraw of 0.55 mL by cardiac puncture over a 60-sec period (under methoxyflurane-anesthesia). FIG. 21 shows that administration of siRNA prior to inducement of hemorrhage shock, provided a protective benefit by decreasing expression of I1-1β and TNF-α.

Thus, one embodiment of the present invention provides a method for decreasing expression of pro-inflammatory cytokines in vivo in a subject, comprising administering eIF5A1 siRNA to the subject, whereby the eIF5A1 siRNA decreases expression of pro-inflammatory cytokines. The subject may be any animal including a human.

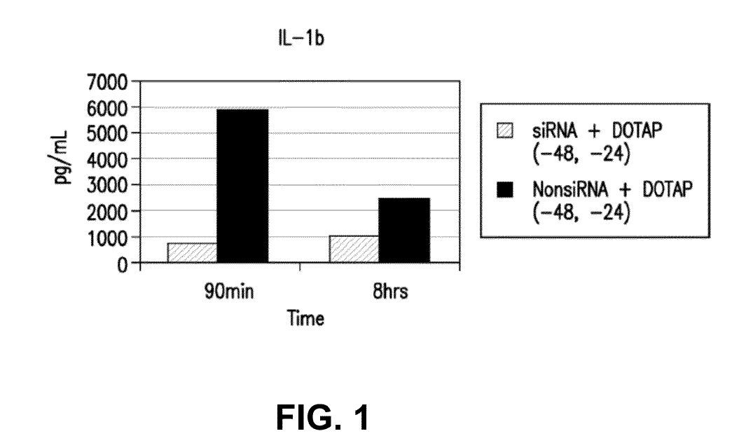

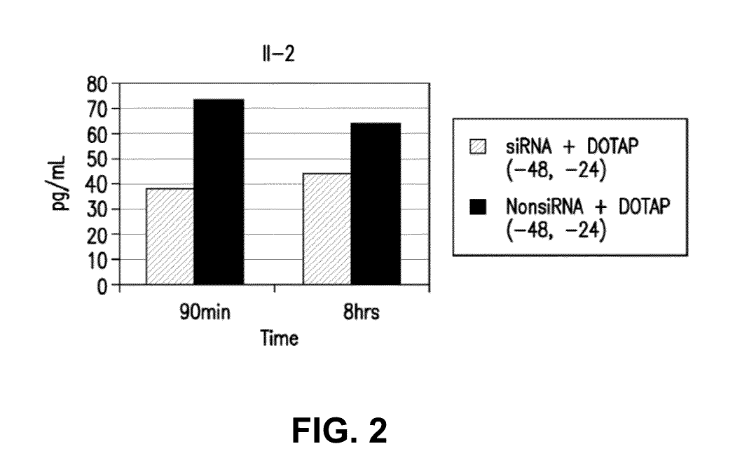

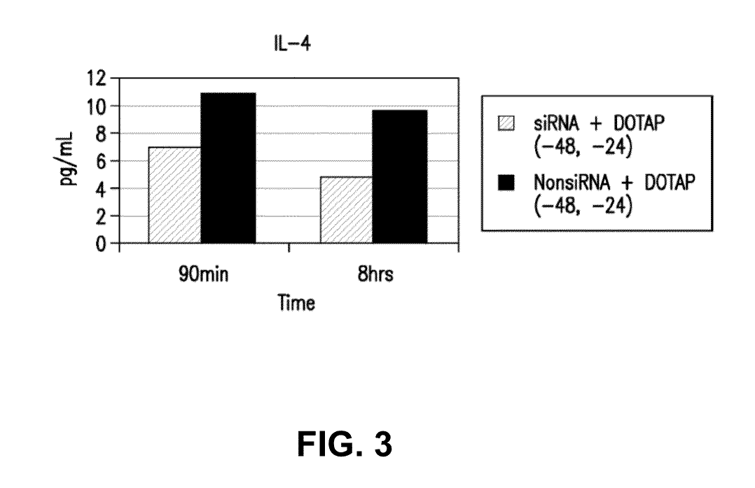

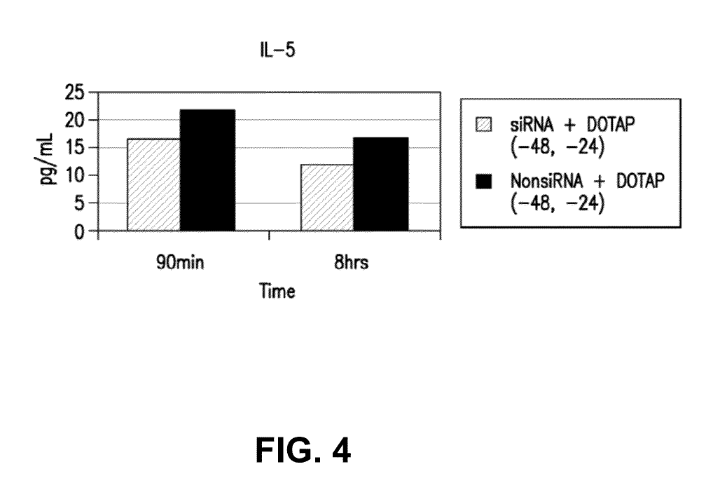

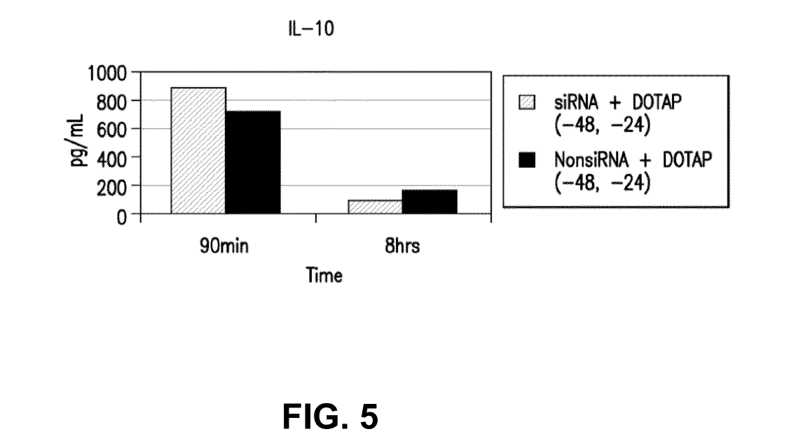

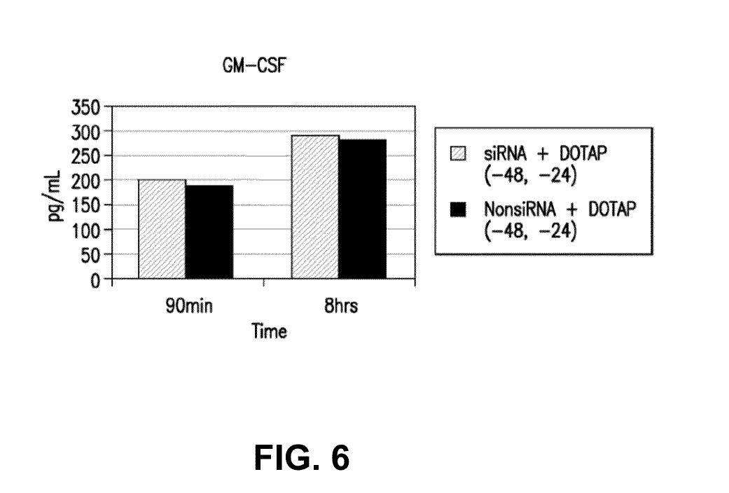

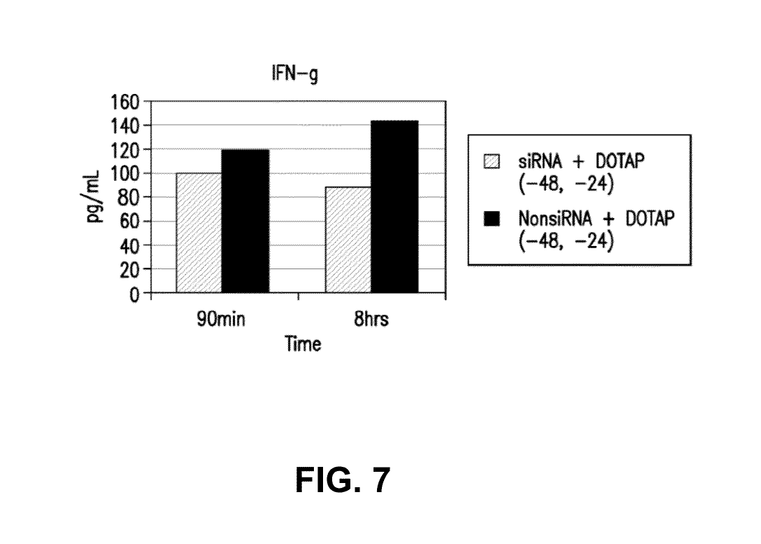

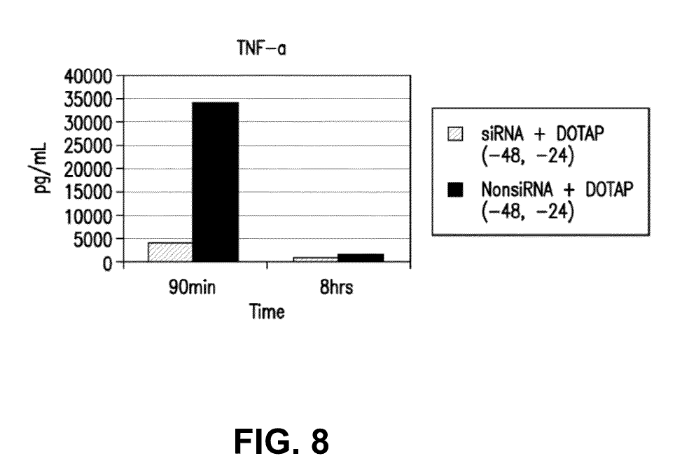

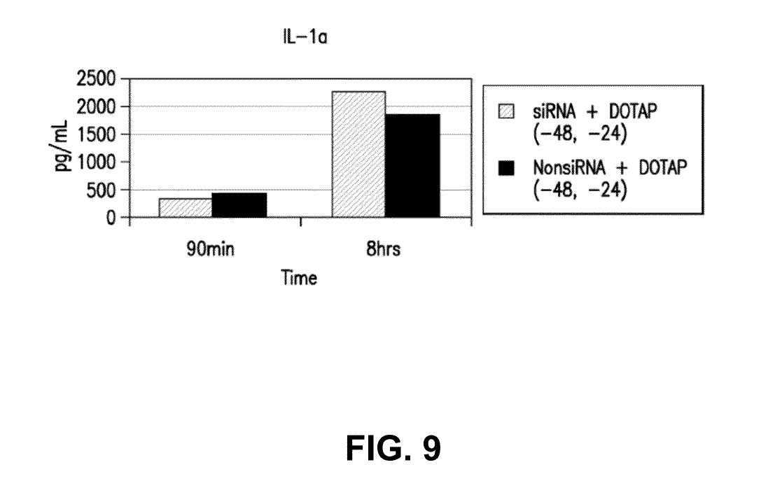

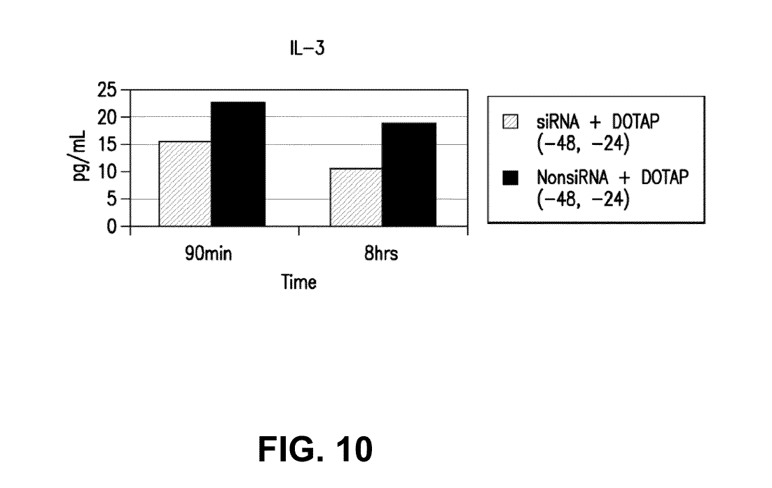

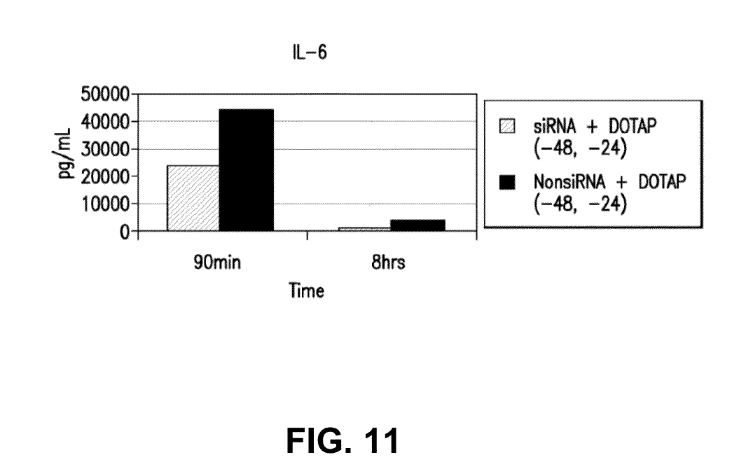

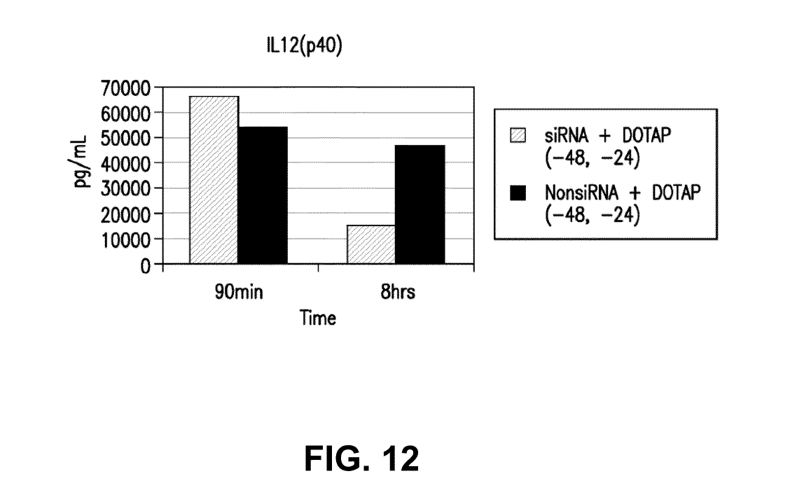

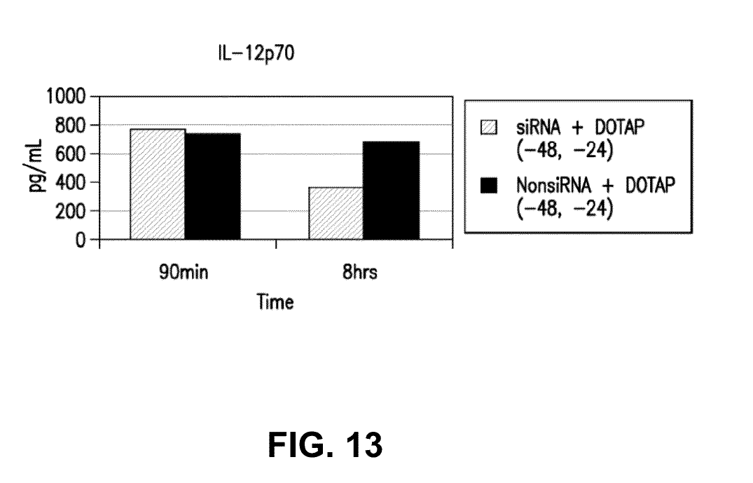

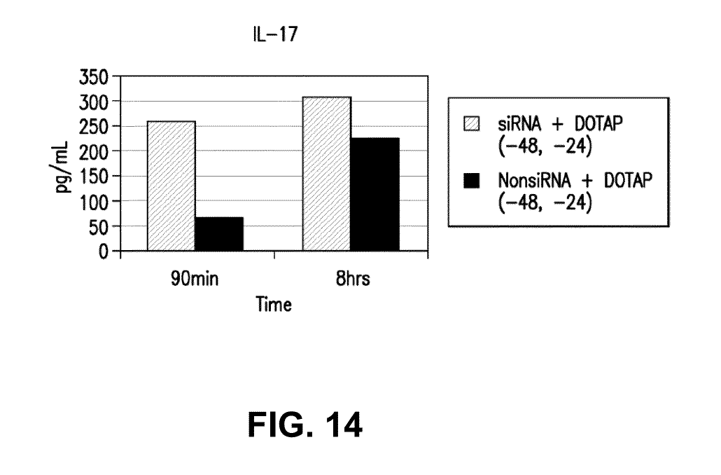

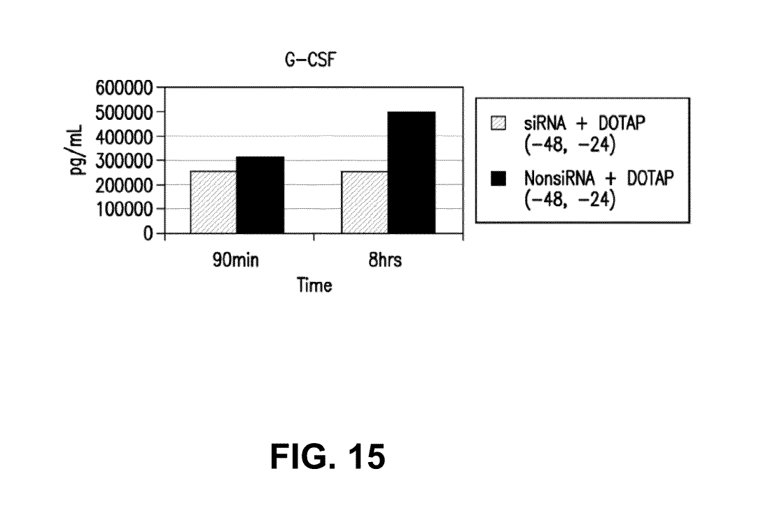

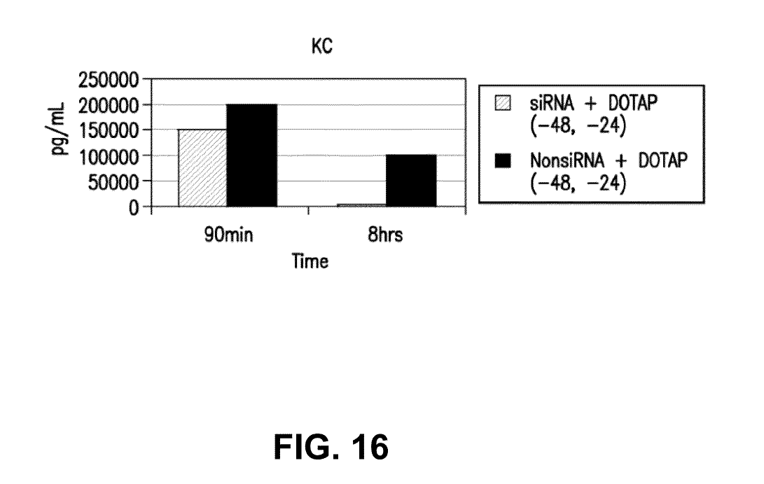

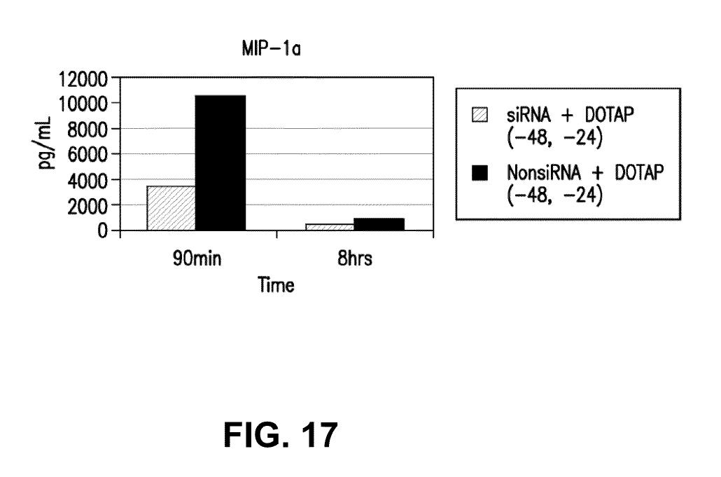

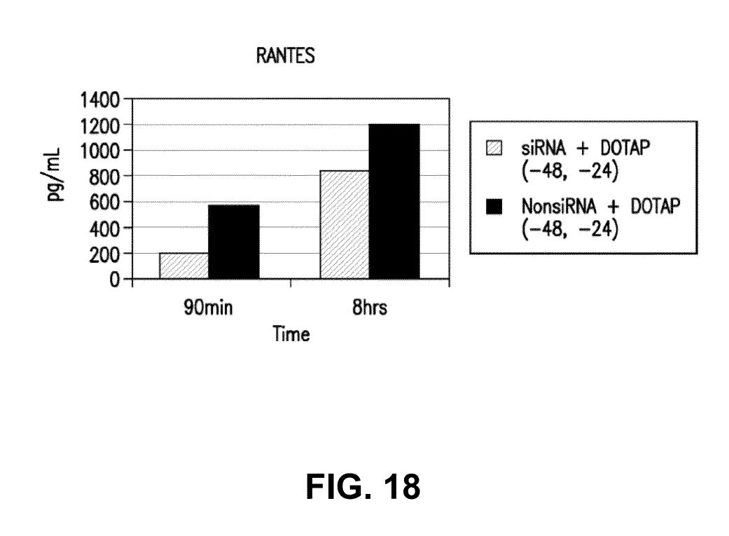

The pro-inflammatory cytokine is any cytokine that is involved in the inflammation cascade, such as IL-1β, IL-2, IL-4, IL-5, IL-10, IFN-γ, TNF-α, IL-3, IL-6, IL-12(p40), IL-12(p70), G-CSF, KC, MIP-1a, and RANTES. FIGS. 1-18 and 21-22 show that treatment with eIF5A1 siRNA resulted in a decreased amount of proinflammatory cytokines as compared to animals not having received the eIF5A1 siRNA.

As shown above, the inventors demonstrated that eIF5A siRNA confers protection in endotoxemic mice when pro-inflammatory cytokine expression was reduced. Hence, one embodiment of the invention also provides a method of treating sepsis in a subject by administering eIF5A1 siRNA to the subject, whereby administration of eIF5A1 siRNA decreases expression of eIF5A1 and results in decreased expression of pro-inflammatory cytokines. Decreased expression means reduced expression as well as decreased or reduced levels of a particular protein as compared to levels of expression or amounts of a protein in a subject not having been treated with eIF5A1 siRNA other eIF5A1 antisense constructs.

Another embodiment of the present invention further provides a method of preventing hemorrhagic shock in a subject, including a human, comprising administering an eIF5A1 siRNA or antisense polynucleotide to decrease expression of IL-1β and/or TNF-α.

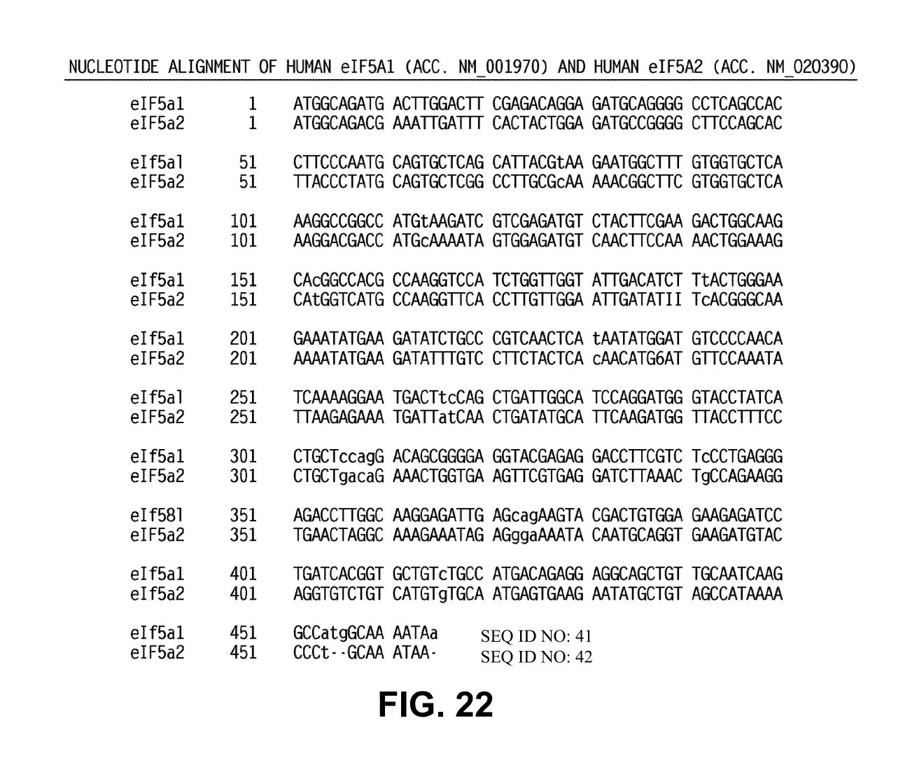

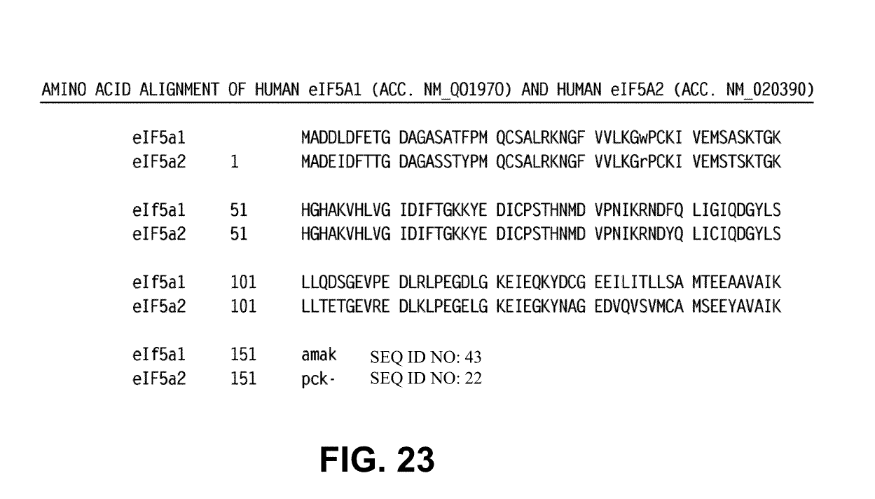

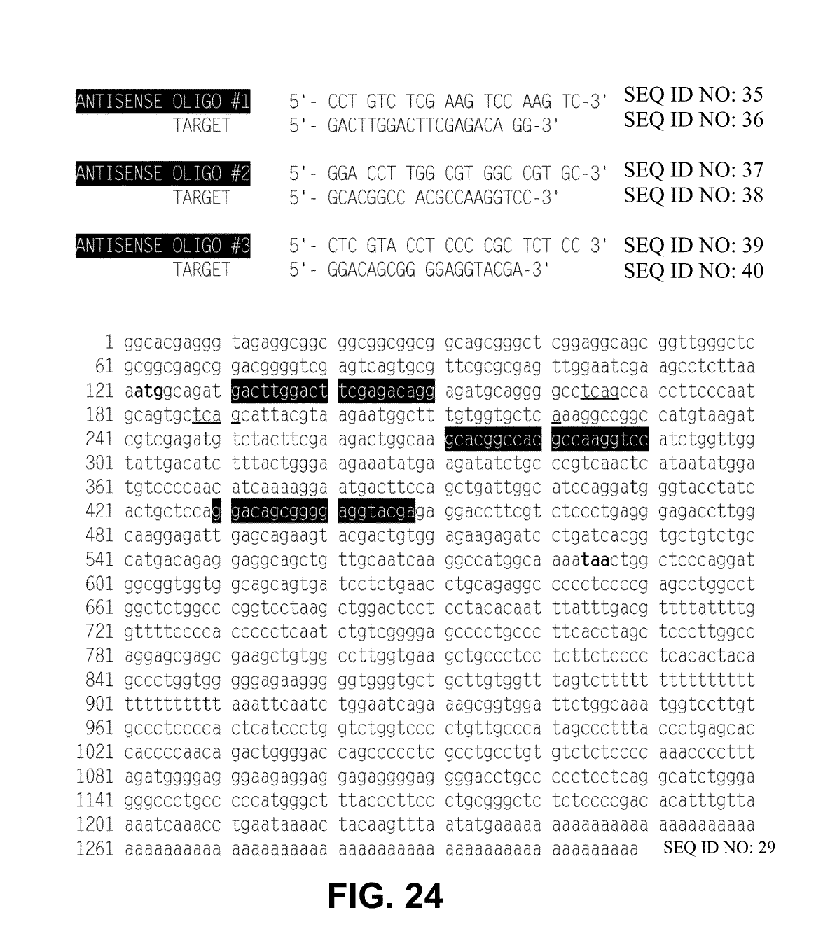

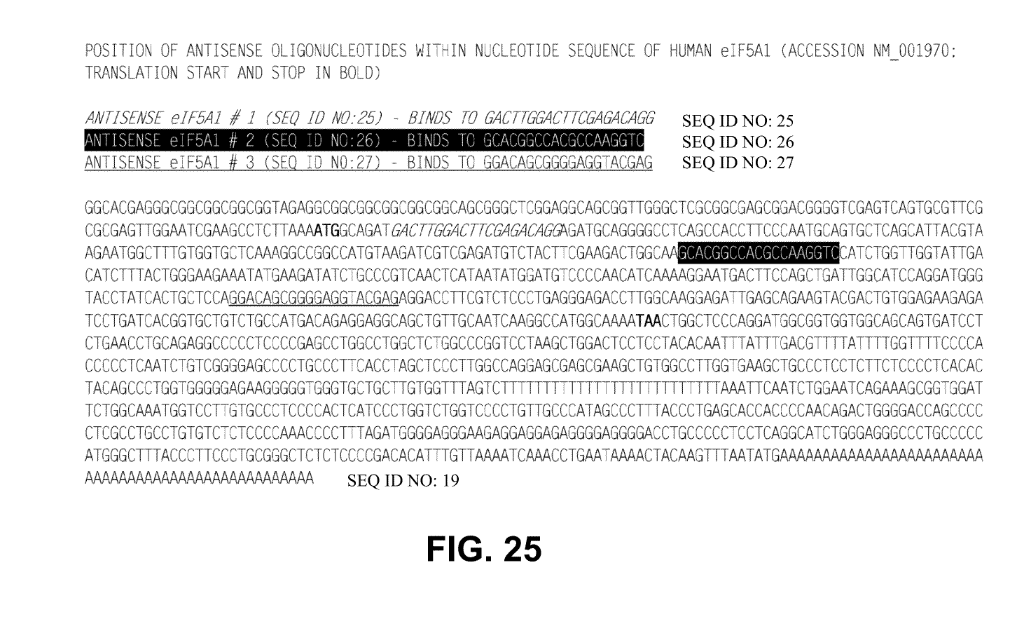

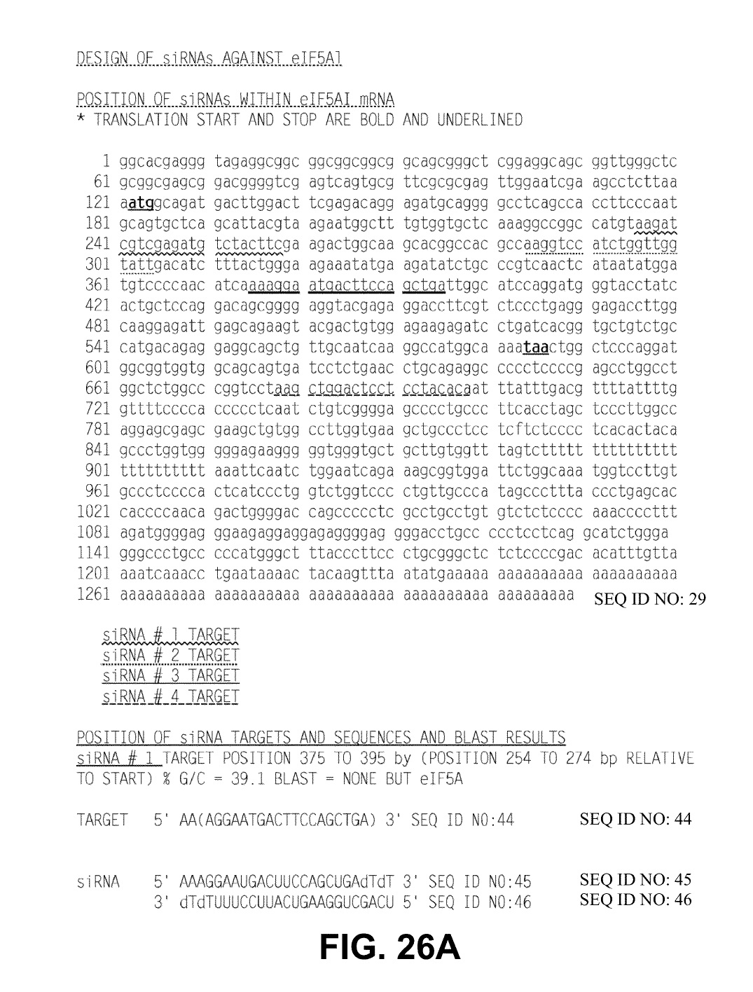

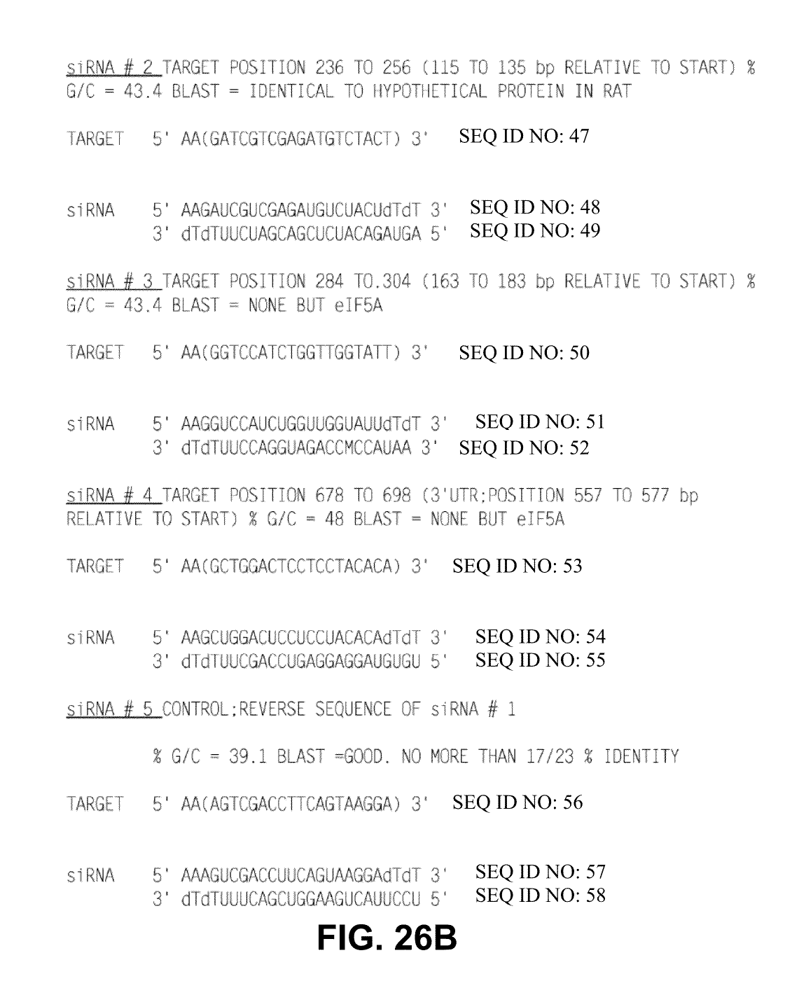

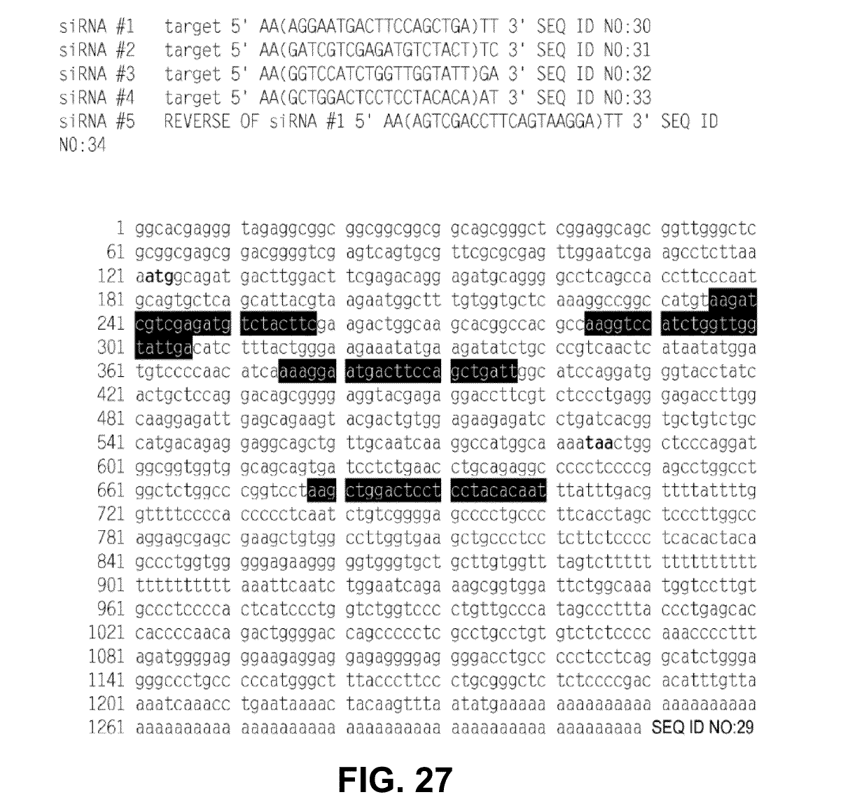

Any eIF5A1 siRNA that inhibits expression of eIF5A1 may be used. The term “inhibits” also means reduce or decrease. One exemplary eIF5A1 siRNA comprises the sequence: CGG AAU GAC UUC CAG CUG A (SEQ ID NO: 117). Co-pending U.S. applications Ser. No. 11/134,445 (filed May 23, 2005), Ser. No. 11/184,982 (filed Jul. 20, 2005), Ser. No. 11/293,391 (filed Nov. 28, 2005), and Ser. No. 11/595,990 (filed Nov. 13, 2006) (which are herein incorporated by reference in its entirety) provides additional exemplary eIF5A1 siRNAs and other antisense constructs that have been used to inhibit expression of eIF5A1 in other cell types and were also shown to inhibit expression of pro-inflammatory cytokines. One skilled in the art could design other eIF5A1 siRNAs given the eIF51A sequence and can easily test for the siRNAs ability to inhibit expression without undue experimentation. FIGS. 22-27 provide sequences of eIF5A1, exemplary eIF5A1 siRNAs and antisense constructs.

The preset invention also provides pharmaceutical compositions comprising eIF-5A1 siRNA or antisense polynucleotides discussed above useful for decreasing expression of pro-inflammatory cytokines. The composition may comprising eIF5A1 siRNA or antisense polynucleotides and a pharmaceutically acceptable carrier. Pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Generally, an effective amount of the eIF5A1 siRNA or eIF5A1 antisense nucleotides described above will be determined by the age, weight and condition or severity of disease of the recipient. Dosing may be one or more times daily, or less frequently. It should be noted that the present invention is not limited to any dosages recited herein.

Pharmaceutical compositions may be prepared as medicaments to be administered in any method suitable for the subject's condition, for example, orally, parenterally (including subcutaneous, intramuscular, and intravenous), rectally, transdermally, buccally, or nasally, or may be delivered to the eye as a liquid solution.

The siRNA or antisense construct can be delivered as “naked” siRNA or antisense nucleotide or may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nanoparticles and nanocapsules), or in macroemulsions. Such techniques are disclosed in Remington's Pharmaceutical Sciences, 16th edition, Oslo, A., Ed., (1980).

The antisense polynucleotides and/or siRNA may be chemically modified. This may enhance their resistance to nucleases and may enhance their ability to enter cells. For example, phosphorothioate oligonucleotides may be used. Other deoxynucleotide analogs include methylphosphonates, phosphoramidates, phosphorodithioates, N3′P5′-phosphoramidates and oligoribonucleotide phosphorothioates and their 2′-O-alkyl analogs and 2′-O-methylribonucleotide methylphosphonates.

Alternatively mixed backbone oligonucleotides (MBOs) may be used. MBOs contain segments of phosphothioate oligodeoxynucleotides and appropriately placed segments of modified oligodeoxy-or oligoribonucleotides. MBOs have segments of phosphorothioate linkages and other segments of other modified oligonucleotides, such as methylphosphonate, which is non-ionic, and very resistant to nucleases or 2′-O-alkyloligoribonucleotides.

Further, the present invention provides a method of treating pathological conditions characterized by an increased IL-1, TNF-alpha, IL-6 or IL-18 level comprising administering to a mammal having said pathological condition, agents to reduce expression of apoptosis-specific eIF-5A as described above (antisense oligonucleotides and siRNA). Known pathological conditions characterized by an increase in IL-1, TNF-alpha, or Il-6 levels include, but are not limited to, arthritis-rheumatoid and osteo arthritis, asthma, allergies, arterial inflammation, crohn's disease, inflammatory bowel disease, (ibd), ulcerative colitis, coronary heart disease, cystic fibrosis, diabetes, lupus, multiple sclerosis, graves disease, periodontitis, glaucoma and macular degeneration, ocular surface diseases including keratoconus, organ ischemia-heart, kidney, repurfusion injury, sepsis, multiple myeloma, organ transplant rejection, psoriasis and eczema. For example, inflammatory bowel disease is characterized by tissue damage caused, in part, by pro-inflammatory cytokines and chemokines released by intestinal epithelial cells.

The present invention also provides a method of delivering siRNA to mammalian lung cells in vivo. siRNAs directed against apoptosis-specific eIF-5A were administered intranasally (mixed with water) to mice. 24 hours after administration of the siRNA against apoptosis-specific eIF-5A, lipopolysaccharide (LPS) was administered intranasally to the mice. LPS is a macromolecular cell surface antigen of bacteria that when applied in vivo triggers a network of inflammatory responses. Intranasally delivering LPS causes an increase in the number of neutrophils in the lungs. One of the primary events is the activation of mononuclear phagocytes through a receptor-mediated process, leading to the release of a number of cytokines, including TNF-α. In turn, the increased adherence of neutrophils to endothelial cells induced by TNF-α leads to massive infiltration in the pulmonary space.

After another 24 hours, the right lung was removed and myeloperoxidase was measured. Myeloperoxidase (“MPO”) is a lysosomal enzyme that is found in neutrophils. MPO uses hydrogen peroxidase to convert chloride to hypochlorous acid. The hypochlorous acid reacts with and destroys bacteria. Myeloperoxidase is also produced when arteries are inflamed. Thus, it is clear that myeloperoxidase is associated with neutrophils and the inflammation response. The mouse apoptosis-specific eIF-5A siRNA suppressed myeloperoxidase by nearly 90% as compared to the control siRNA. In the study, there were 5 mice in each group. The results of this study show that siRNA can be delivered successfully in vivo to lung tissue in mammals, and that siRNA directed against apoptosis-specific eIF-5A inhibits the expression of apoptosis-specific eIF-5A resulting in a suppression of myeloperoxidase production.

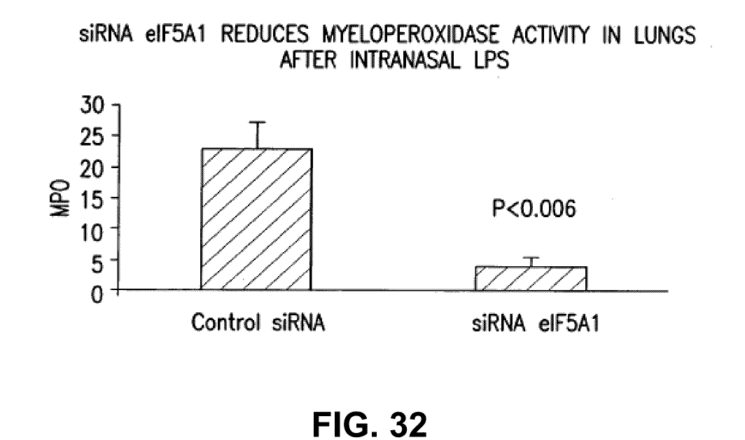

The present inventors have thus demonstrated that down regulating apoptosis-specific eIF-5A with siRNAs decreases levels of myeloperoxidase in lung tissue after exposure to LPS (which normally produces an inflammatory response involving the production of myeloperoxidase), and thus decrease or suppress the inflammation response. See FIG. 32 showing that after mice received LPS and eIF-5A1 siRNA intranasaly they had a reduced myeloperoxidase activity as compared to mice receiving control siRNA. Accordingly, one embodiment of the present invention provides a method of reducing levels of MPO in lung tissue by delivering siRNAs against apoptosis-specific eIF-5A to inhibit or reduce expression of apoptosis-specific eIF-5A. The reduction in the expression of apoptosis-specific eIF-5A leads to a reduction of MPO. Delivery of the siRNA apoptosis-specific eIF-5A may be intranasal.

MPO levels are a critical predictor of heart attacks and cytokine-induced inflammation caused by autoimmune disorders. This ability to decrease or suppress the inflammation response may serve useful in treating inflammation related disorders such as auto-immune disorders. In addition, the ability to lower MPO could be a means of protecting patients from ischemic events and heart attacks.

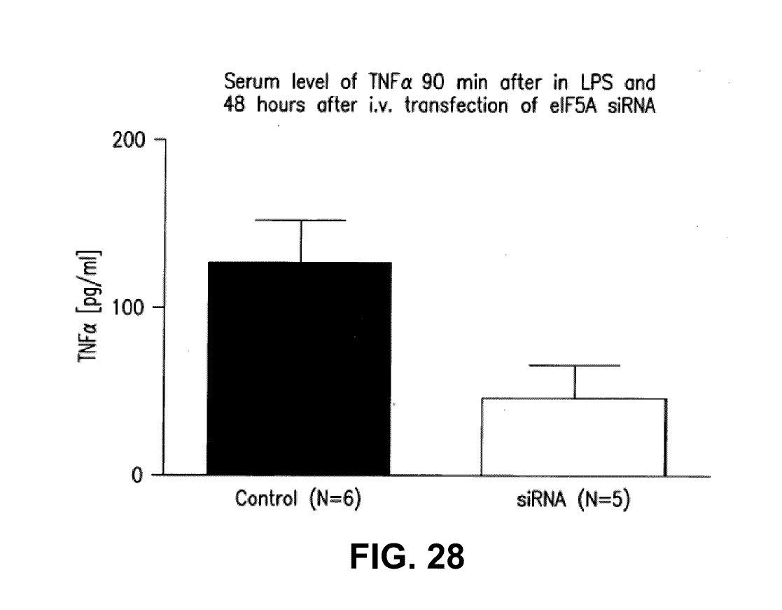

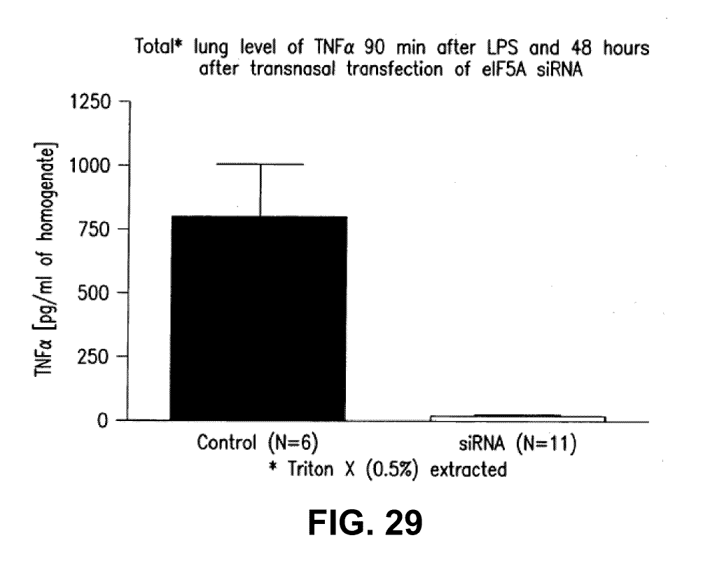

FIG. 28 shows the results of an experiment performed in mice where siRNAs against apoptosis-specific eIF-5A were able to decrease the level of TNF-α in the mice serum. The siRNAs were delivered intravenously into a tail vein of the mice. The TNFα serum levels were measured 90 minutes after administration of LPS and 48 hours after intravenous transfection of siRNAs against apoptosis-specific eIF-5A. FIG. 29 shows the results of an experiment performed in mice where the siRNAs were delivered trans-nasally (as described above). Total levels of TNF-α were measured in the serum of the mice. The siRNAs against apoptosis-specific eIF-5A caused a decrease in the amount of TNFα. Accordingly, one embodiment of the present invention provides a method of reducing levels of TNF-α in serum by delivering siRNAs against apoptosis-specific eIF-5A to inhibit or reduce expression of apoptosis-specific eIF-5A. The reduction in the expression of apoptosis-specific eIF-5A leads to a reduction of TNF-α in the serum.

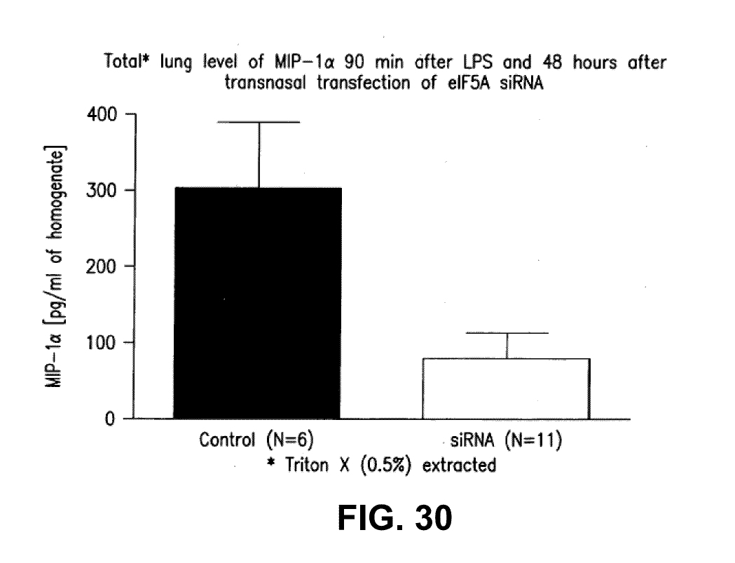

FIG. 30 shows that levels of macrophage inflammatory protein 1-alpha (MIP-1α) were also decreased. MIP-1α is a low molecular weight chemokine that belongs to the RANTES (regulated on activation normal T cell expressed and secreted) family of cytokines and binds to receptors CCR1, CCR5 and CCR9. Accordingly, one embodiment of the present invention provides a method of reducing levels of MIP-1α in lung tissue by delivering siRNAs against apoptosis-specific eIF-5A to inhibit or reduce expression of apoptosis-specific eIF-5A. The reduction in the expression of apoptosis-specific eIF-5A leads to a reduction of MIP-1α.

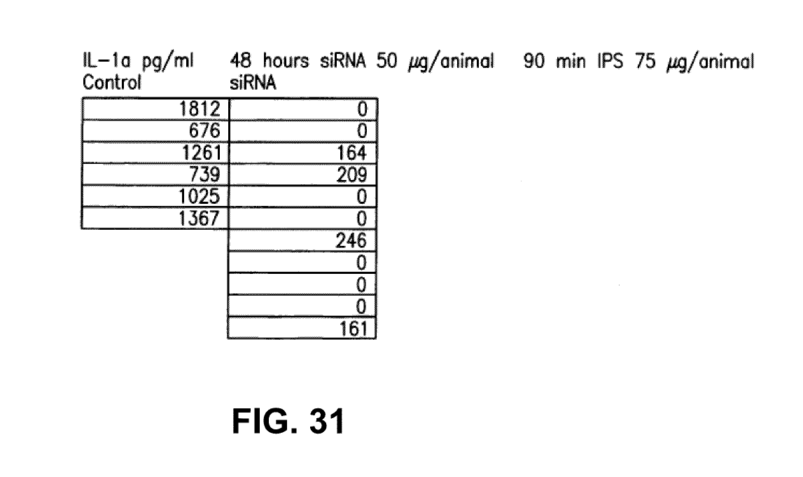

FIG. 31 shows the results of an experiment where mice were treated with siRNAs against apoptosis-specific eIF-5A (intranasal/transnasal delivery). The results show that 90 minutes after treatment with LPS and 48 hours after being treated with the siRNAs, there was a marked decrease in levels of Il-1α measured the mice lungs as compared to mice lungs not having been treated with siRNAs against apoptosis-specific eIF-5A. Accordingly, one embodiment of the present invention provides a method of reducing levels of Il-1α in lung tissue by delivering siRNAs against apoptosis-specific eIF-5A to inhibit or reduce expression of apoptosis-specific eIF-5A. The reduction in the expression of apoptosis-specific eIF-5A leads to a reduction of Il-1α.

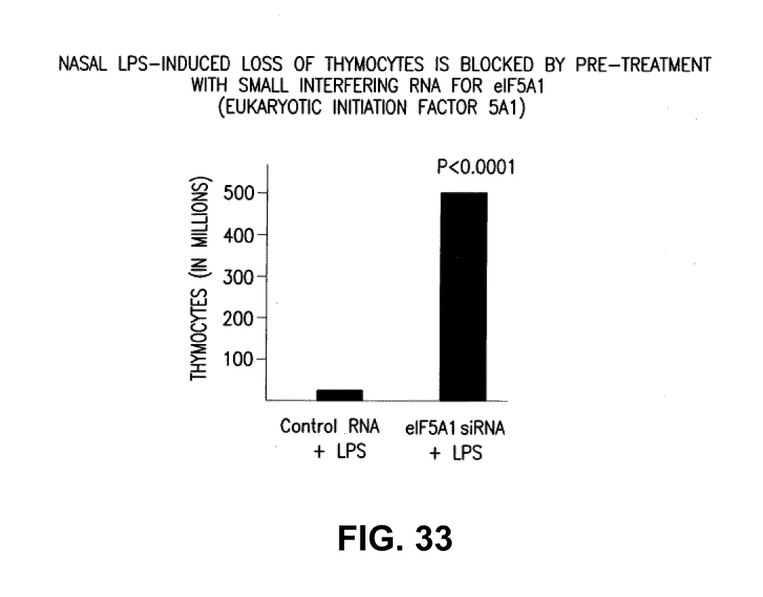

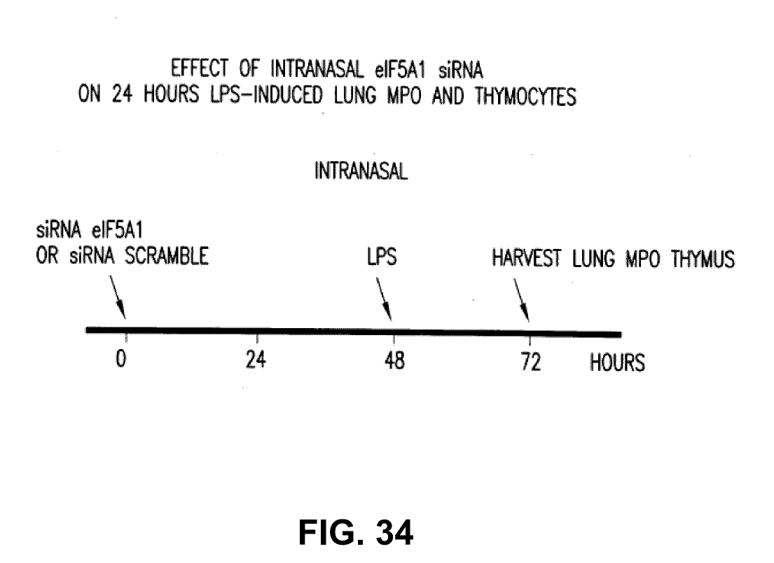

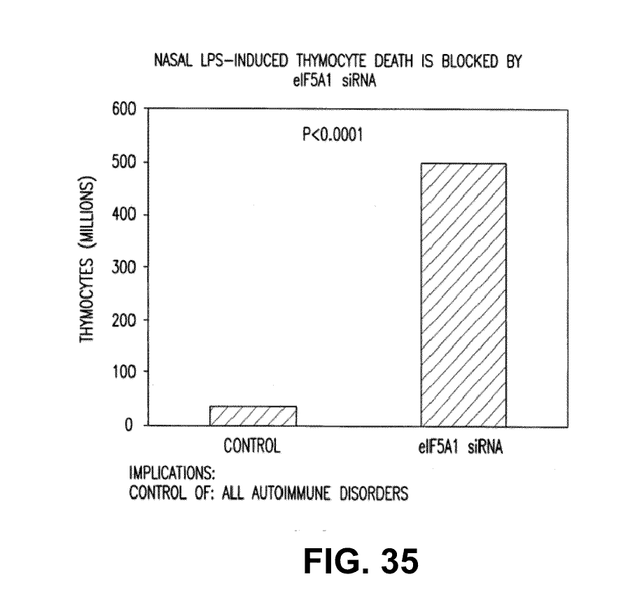

FIGS. 33-35 show that nasal-LPS-induced loss of thymocyes is blocked by pre-treatment with apoptosis-specific eIF-5A siRNA. Accordingly, one method of the present invention provides a method of protecting against LPS-induced thymocyte apoptosis, wherein siRNA against apoptosis-specific eIF-5A is delivered to a mammal intranasaly.

Thymocyte T cell development is a complex event involving distinct stages of proliferation and cell death. Bacterial infections result in the release of bacterial cell wall components such as LPS, lipoteichoic acid, and peptidoglycans. These cell wall components lead to the production of cytokines such as IL-1β, IL-6, IL-8 and TNF-α, each of which contributes to the increased risk of sepsis progressing to sepsis syndrome, shock and death. In animal models of systemic inflammatory conditions, the administration of microbial products such as LPS, thymocyte apoptosis is observed.

Pulmonary infection caused by Gram-negative bacteria activates alveolar macrophages resulting in the production of cytokines such as IL-1 and TNF-α. In turn, these cytokines recruit polymorphonuclear neutrophils into the inflammatory site and in late stages of severe infection, septic shock may develop. Increasing evidence suggests that apoptosis occurs in many organs during sepsis, including the thymus. Thus, the effect of intranasal LPS administration on thymocyte apoptosis was studied. The results of the study show that mice treated with LPS intranasally have reduced thymus cellularity. Thymic cellularity was significantly lower 24 hours after intranasal LPS and returned to control levels after 48 hours. Similarly, peak apoptosis was observed 24 hours after LPS administration (32%) and recovered by 48 hours. These observations are similar to what observed after intraperitoneal injection of LPS, where peak apoptosis was reached 24 hours after LPS administration (28%) as well as what we have previously observed after intravenous conA injection (46%).

Fas and FasL are expressed in the thymus and LPS-induced thymocyte apoptosis is mediated by glucocorticoids, which is in turn, increase the expression of Fas/FasL. It is possible that siRNA eIF5A reduced LPS-induced apoptosis by down regulating thymocyte Fas/FasL. In addition, LPS activates NF-kB, which leads to the synthesis and release of a number of proinflammatory mediators, including IL-1, IL-6, IL-8, and TNF-α (37). Because TNF-α and IFN-γ are both critical mediators in thymus atrophy and thymocyte apoptosis induced systemic inflammation, the mechanism by which siRNA inhibits LPS-induced thymocyte apoptosis could be due to lower levels of TNF-α and other proinflammatory cytokines since siRNA eIF-5A strongly inhibits TNF-α production by IFN-γ primited HT-29 cells in response to LPS. Therefore, the mechanism by which siRNA eIF-5A suppresses LPS-induced thymocyte apoptosis could be the result of decreased synthesis of TNF-α and IFN-γ, indicating that eIF-5A may be an important target for the development of anti-inflammatory therapeutics.

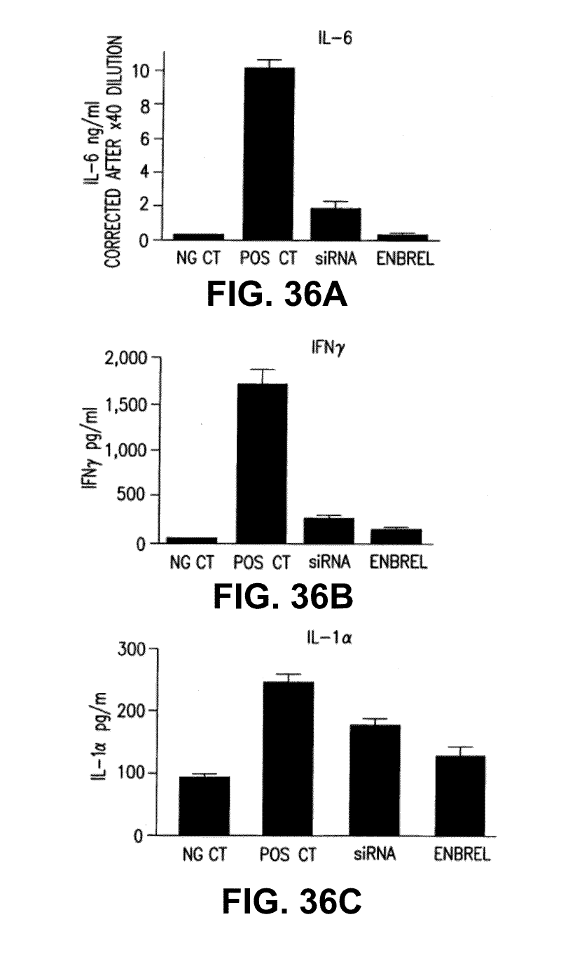

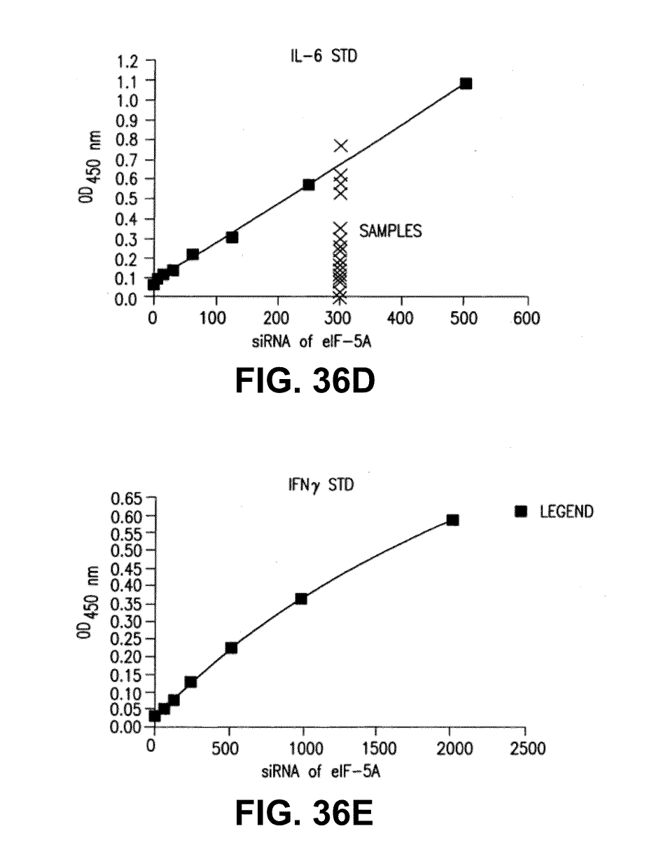

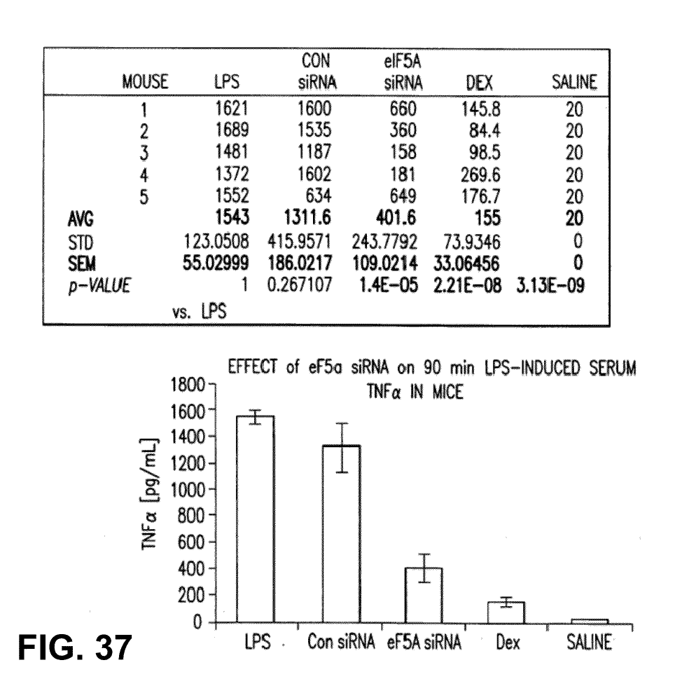

FIGS. 36A-E show that siRNA against eIF-5A delivered intranasaly decreased production of IL-6, IFN-γ and Il-1α in mice. FIG. 37 shows that siRNA against eIF-5A is able to reduce the expression of TNFα as a result of treatment with LPS. The top panel shows the raw data and the bottom panel shows the data in a bar graph.

Thus, the present inventors shown the correlation between apoptosis-specific eIF-5A and the immune response, as well as shown that siRNAs against apoptosis-specific eIF-5A suppress the production of myeloperoxidase (i.e. part of the inflammation response). The inventors have also shown that it is possible to deliver siRNAs in vivo to lung tissue by simple intranasal delivery. The siRNAs were mixed only in water. This presents a major breakthrough and discovery as others skilled in the art have attempted to design acceptable delivery methods for siRNA.

In another experiment, mice were similarly treated with siRNAs directed against apoptosis-specific eIF-5A. Lipopolysaccharide (LPS) was administered to the mice to induce inflammation and an immune system response. Under control conditions, LPS kills thymocytes, which are important immune system precursor cells created in the thymus to fend off infection. However, using the siRNAs directed against apoptosis-specific eIF-5A allowed approximately 90% survivability of the thymocytes in the presence of LPS. When thymocytes are destroyed, since they are precursors to T cells, the body's natural immunity is compromised by not being able to produce T cells and thus can't ward off bacterial infections and such. Thus, siRNAs against apoptosis-specific eIF-5A can be used to reduce inflammation (as shown by a lower level of MPO in the first example) without destroying the body's natural immune defense system.

Another embodiment of the present invention provides a method to treat sepsis by administering siRNA against apoptosis-specific eIF-5A. Sepsis is also known as systemic inflammatory response syndrome (“SIRS”). Sepsis is caused by bacterial infection that can originate anywhere in the body. Sepsis can be simply defined as a spectrum of clinical conditions caused by the immune response of a patient to infection that is characterized by systemic inflammation and coagulation. It includes the full range of response from systemic inflammatory response (SIRS) to organ dysfunction to multiple organ failure and ultimately death.

Sepsis is a very complex sequence of events and much work still needs to be done to completely understand how a patient goes from SIRS to septic shock. Patients with septic shock have a biphasic immunological response. Initially they manifest an overwhelming inflammatory response to the infection. This is most likely due to the pro-inflammatory cytokines Tumor Necrosis Factor (TNF), IL-1, IL-12, Interferon gamma (IFNgamma), and IL-6. The body then regulates this response by producing anti-inflammatory cytokines (IL-10), soluble inhibitors [TNF receptors, IL-1 receptor type II, and IL-1RA (an inactive form of IL-1)], which is manifested in the patient by a period of immunodepression. Persistence of this hyporesponsiveness is associated with increased risk of nosocomial infection and death.

This systemic inflammatory cascade is initiated by various bacterial products. These bacterial products (gram-negative bacteria=endotoxin, formyl peptides, exotoxins, and proteases; gram-positive bacteria=exotoxins, superantigens (toxic shock syndrome toxin (TSST), streptococcal pyrogenic exotoxin A (SpeA)), enterotoxins, hemolysins, peptidoglycans, and lipotechoic acid, and fungal cell wall material) bind to cell receptors on the host's macrophages and activate regulatory proteins such as Nuclear Factor Kappa B (NFkB). Endotoxin activates the regulatory proteins by interacting with several receptors. The CD receptors pool the LPS-LPS binding protein complex on the surface of the cell and then the TLR receptors translate the signal into the cells.

The pro-inflammatory cytokines produced are tumor necrosis factor (TNF), Interleukins 1, 6 and 12 and Interferon gamma (IFNgamma). These cytokines can act directly to affect organ function or they may act indirectly through secondary mediators. The secondary mediators include nitric oxide, thromboxanes, leukotrienes, platelet-activating factor, prostaglandins, and complement. TNF and IL-1 (as well as endotoxin) can also cause the release of tissue-factor by endothelial cells leading to fibrin deposition and disseminated intravascular coagulation (DIC).

Then these primary and secondary mediators cause the activation of the coagulation cascade, the complement cascade and the production of prostaglandins and leukotrienes. Clots lodge in the blood vessels which lowers profusion of the organs and can lead to multiple organ system failure. In time this activation of the coagulation cascade depletes the patient's ability to make clot resulting in DIC and ARDS.

The cumulative effect of this cascade is an unbalanced state, with inflammation dominant over antiinflammation and coagulation dominant over fibrinolysis. Microvascular thrombosis, hypoperfusion, ischemia, and tissue injury result. Severe sepsis, shock, and multiple organ dysfunction may occur, leading to death.

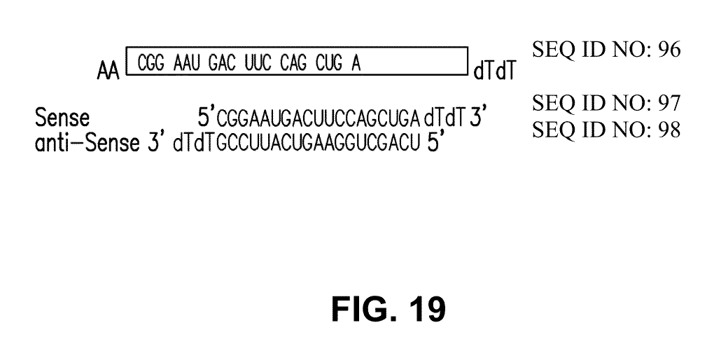

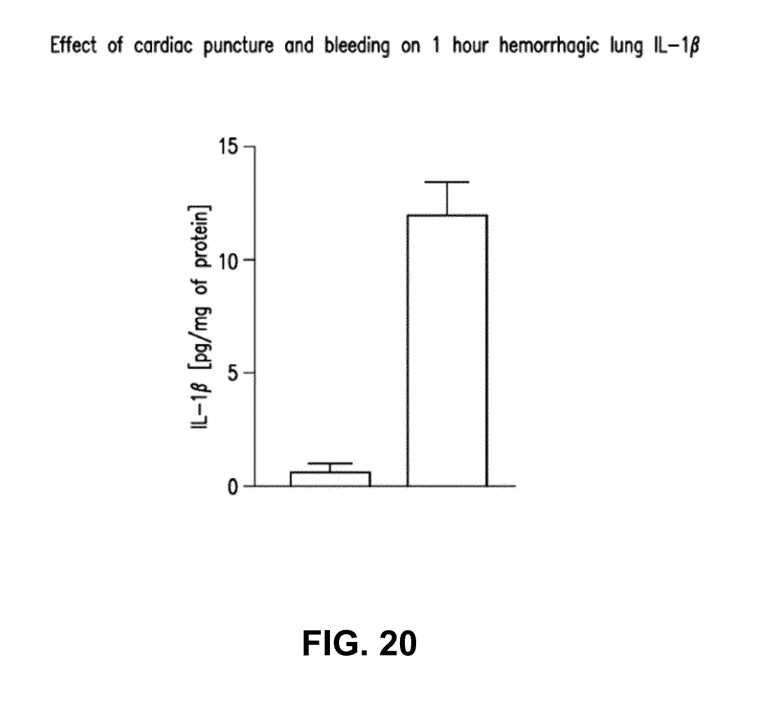

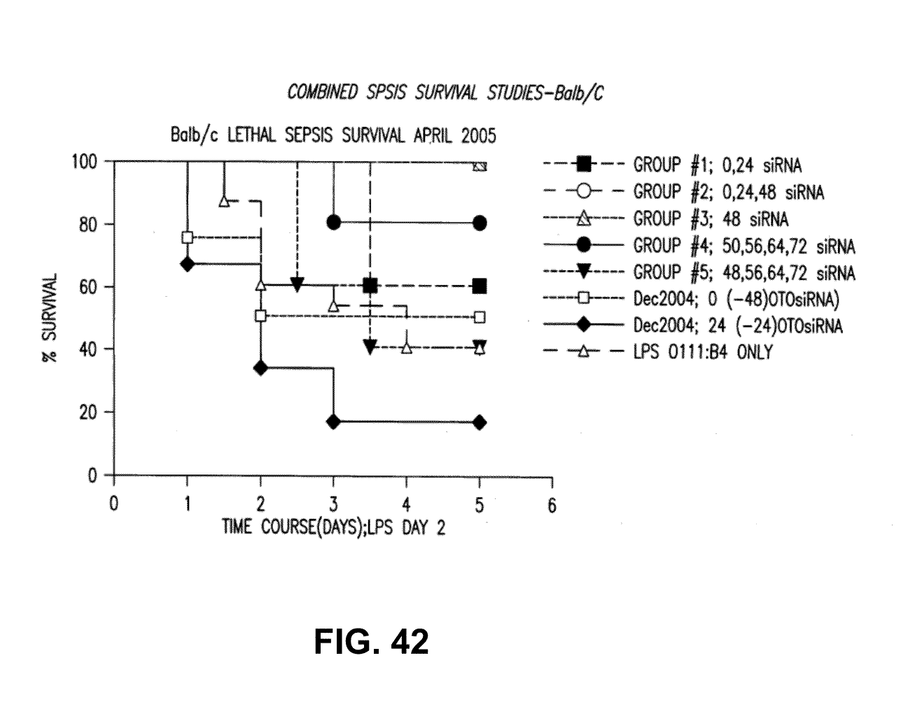

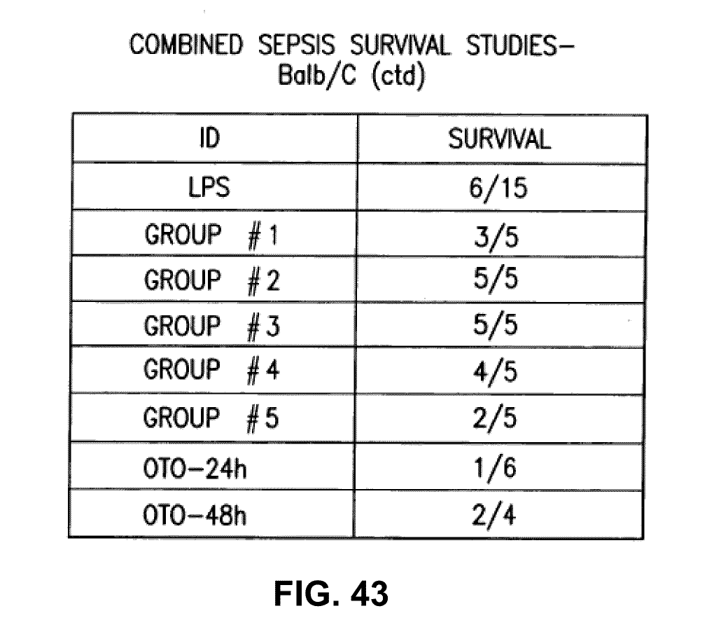

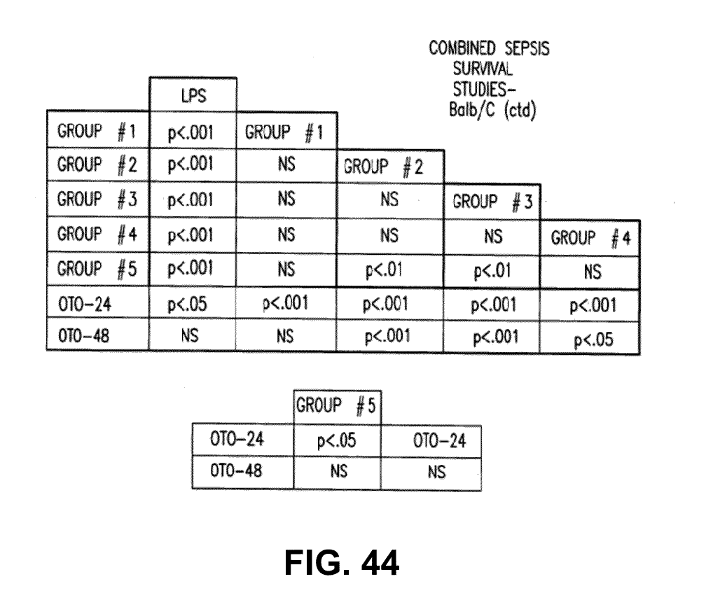

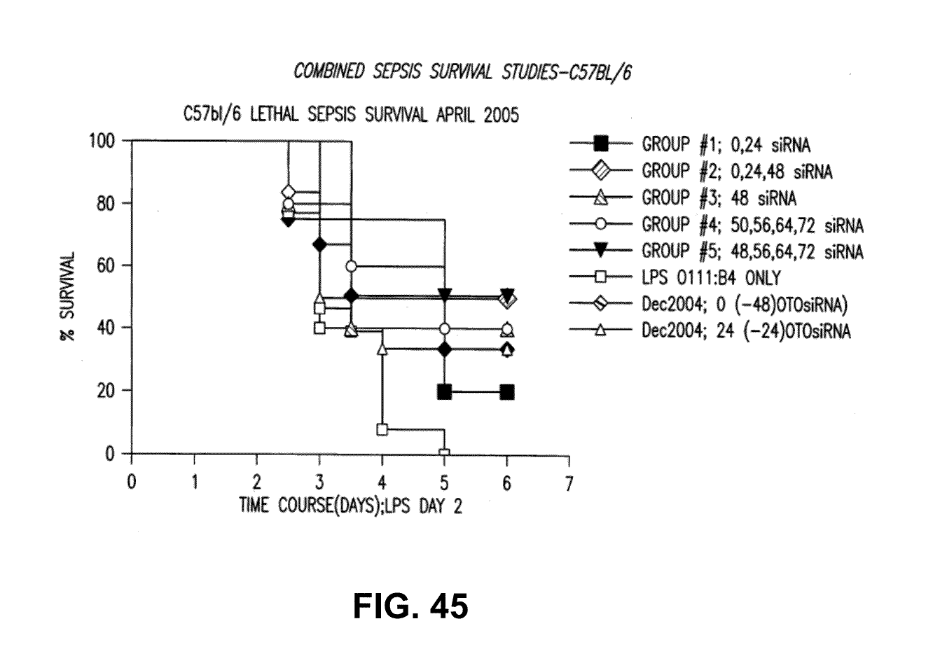

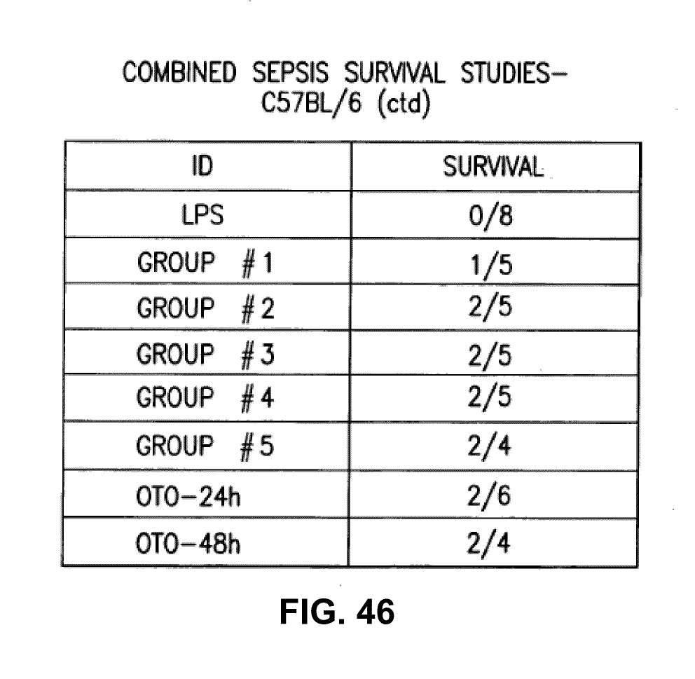

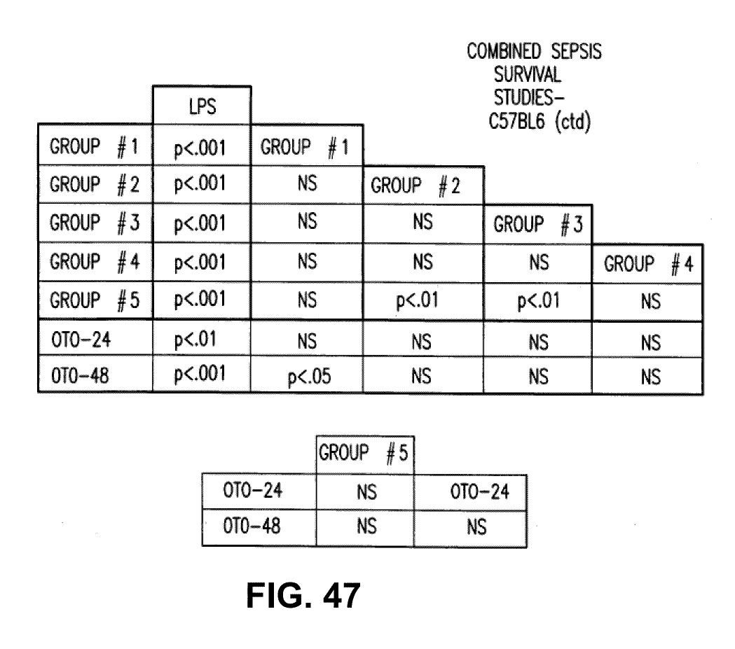

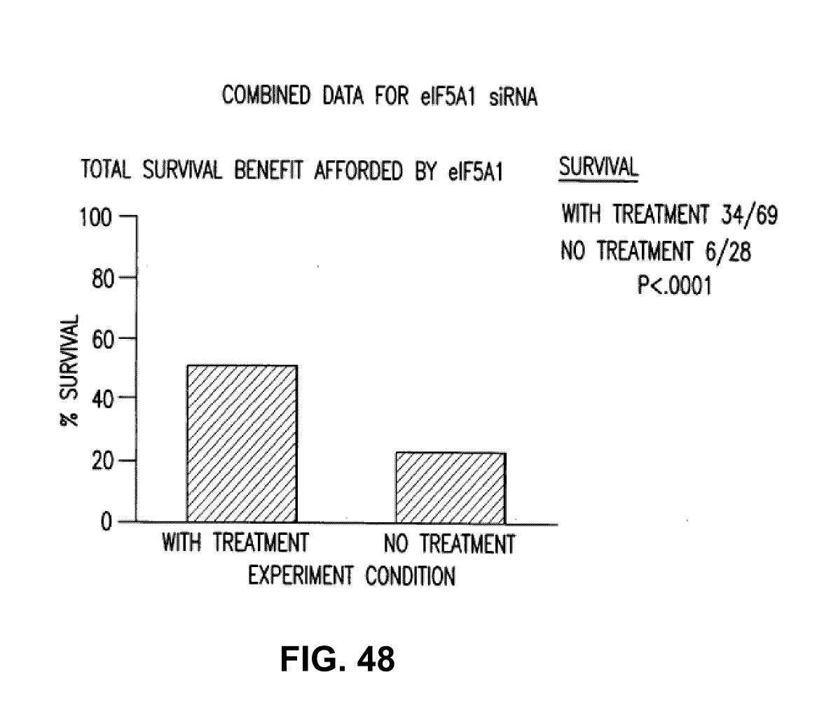

The inventors have previously shown (and presented above) that siRNA against eIF-5A was able to reduce the expression of various inflammation cytokines, such as TNF-α. In a study that involved administering siRNA against apoptosis-specific eIF-5A to treat sepsis in mice, the present inventors have further shown that the siRNA can be used to treat sepsis in vivo. See FIGS. 19 and 38-48. FIG. 19 shows the construct of the siRNA used in the septic mice models. In this study, the mice were given a dose of LPS that induces sepsis and death in the animal within 48 hours after the LPS is administered. siRNA (3′-GCC UUA CUG AAG GUC GAC U-5′; SEQ ID NO: 99) was administered intraperitoneally to mice at different time periods before and after LPS administration. In some test groups, all five mice who received siRNA survived. It is believed that the use of siRNA was able to shut down the inflammation cascade and thus prevent sepsis in the mice.

Accordingly, one embodiment of the present invention provides a siRNA oligonucleotide of apoptosis-specific eIF-5A wherein said siRNA oligonucleotide suppresses endogenous expression of apoptosis-specific eIF-5A in a cell and having the sequence of 3′-GCC UUA CUG AAG GUC GAC U-5′ (SEQ ID NO: 99). By suppressing expression of apoptosis-specific eIF-5A, the production of inflammatory cytokines is inhibited or reduced such that the inflammation cascade does not begin and result in septic shock.

The apoptosis-specific eIF-5A is believed to shuttle subsets of mRNA out of the nucleus that are involved in apoptosis and inflammation. If the amount of eIF-5A is reduced or completely eliminated, there is no shuttle available to shuttle mRNAs of various inflammatory and cell death cytokines out of the nucleus. This results in a decreased amount of inflammatory cytokines produced by the cell and thus, inhibits the beginning of the inflammation cascade. Since sepsis and septic shock are a result of the inflammation cascade, shutting down the cascade provides a method of treating or preventing sepsis/septic shock. Accordingly, another embodiment of the present invention provides a method for treating sepsis in a mammal, comprising administering the siRNAs described previously to a mammal.

Treating Sepsis in a mammal-based on mouse septic model: Two types of groups of mice were used in the study. Balb/C mice and C57BL/6 mice were used. In both studies, the mice were given a dose of LPS that would induce sepsis and death within 48 hours 100% of the time. The test was designed so that siRNA against eIF-5A1 (3′-GCC UUA CUG AAG GUC GAC U-5′; SEQ ID NO: 99) was given intraperitoneal at different time courses. All doses of siRNA were 50 μg. In each study, 5 test groups and 1 control group were used. Each group started with 5 mice. The control group received no siRNA.

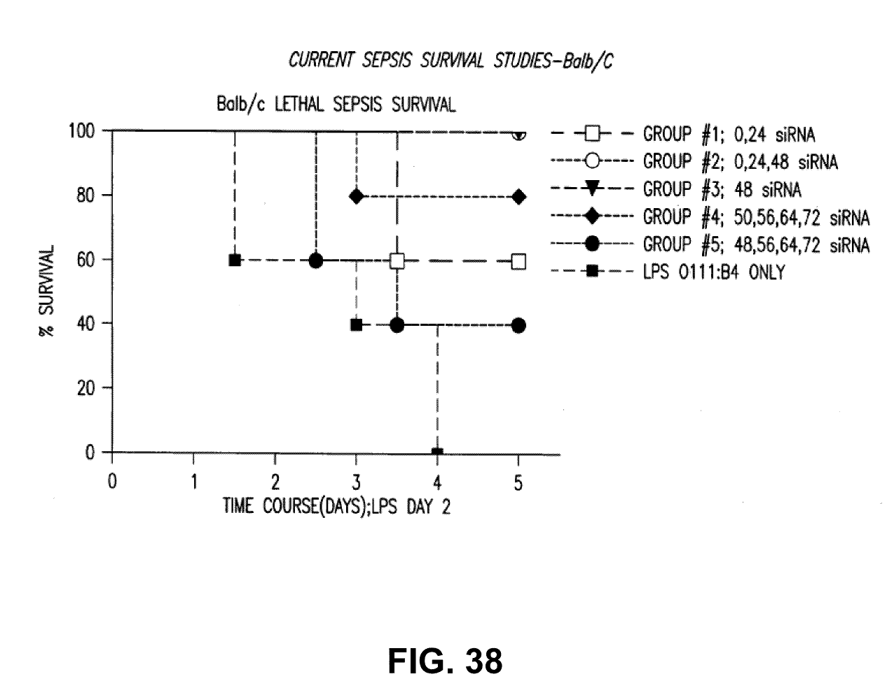

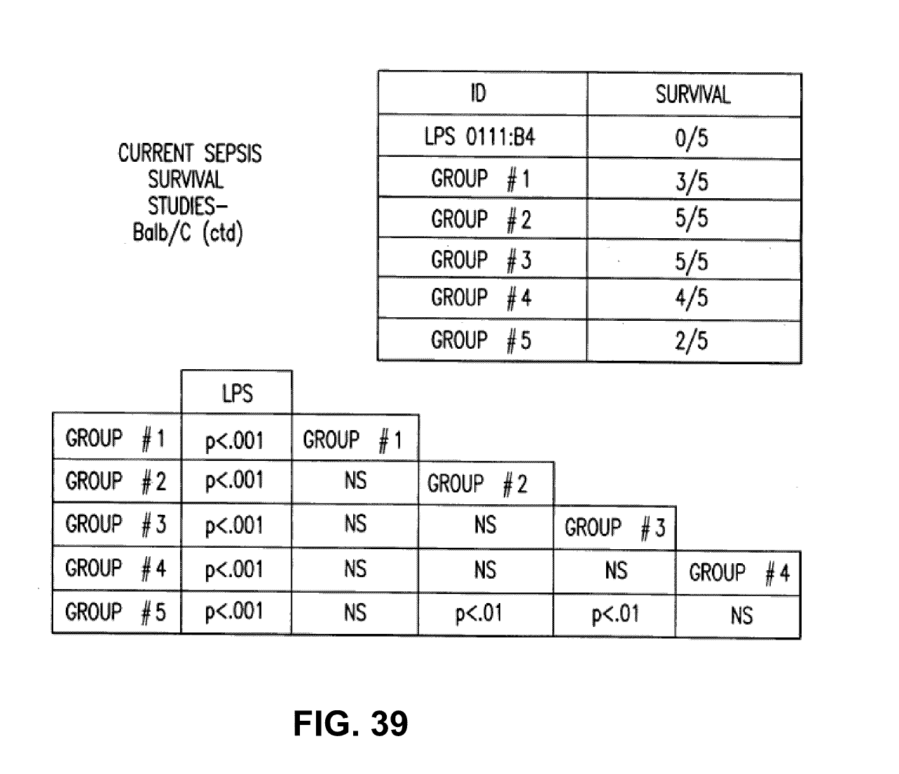

Balb/C mouse model: FIGS. 38 and 39 show the results of the test in Balb/C mice. All mice received the lethal dose of LPS at 48 hours. Group 1 mice received siRNA at 0 and 24 hours, and three out of five mice survived. Group 2 mice received siRNA at 0, 24, and 48 hours, and five out of five mice survived. Group 3 mice received siRNA at 48 hours and five out of five mice survived. Group 4 mice received siRNA at 50, 56, 64 and 72 hours, and four out of five mice survived. Group 5 mice received siRNA at 48, 56, 64 and 72 hours and two out of five mice survived. Group 6 mice, the control group, received no siRNA, and zero mice survived and all five died within 48 hours of LPS treatment (Day 4).

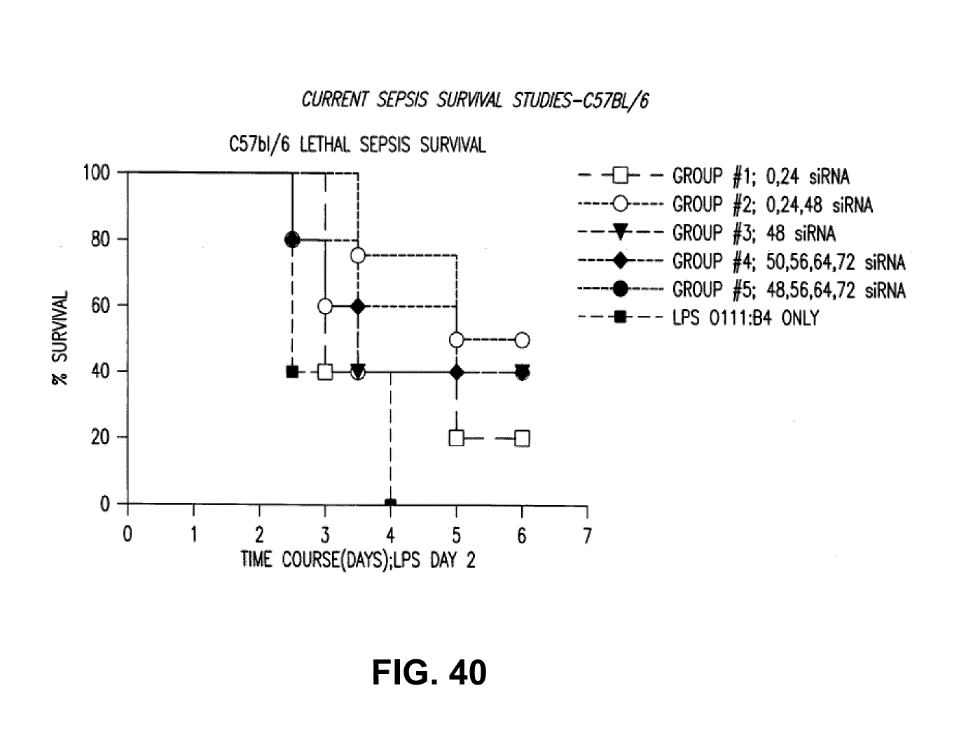

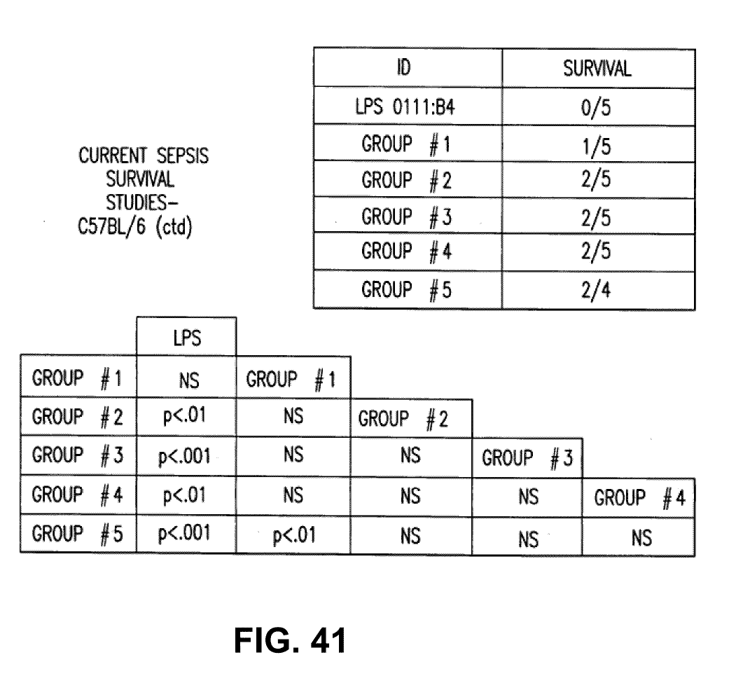

C57BL/6 mouse model: FIGS. 40 and 41 show the results of the test in C57BL/6 mice. All mice received the lethal dose of LPS at 48 hours. Group 1 mice received siRNA at 0 and 24 hours, and one out of five mice survived. Group 2 mice received siRNA at 0, 24, and 48 hours, and two out of five mice survived. Group 3 mice received siRNA at 48 hours and two out of five mice survived. Group 4 mice received siRNA at 50, 56, 64 and 72 hours, and two out of five mice survived. Group 5 mice received siRNA at 48, 56, 64 and 72 hours and two out of five mice survived. Group 6 mice, the control group, received no siRNA, and zero mice survived and all five died within 48 hours of LPS treatment (Day 4).

What is claimed is:

1. A method of treating sepsis in a subject, comprising administering an eIF5A1 siRNA to the subject in an amount sufficient to decrease expression of pro-inflammatory cytokines in the subject, thereby treating sepsis in the subject.

2. The method of claim 1 wherein the subject is a human.

3. The method of claim 1 wherein the pro-inflammatory cytokine is selected from the group consisting of IL-1β, IL-2, IL-4, IL-5, IL-10, IFN-γ, TNF-α, IL-3, IL-6, IL-12(p40), IL-12(p70), G-CSF, KC, MIP-1α, and RANTES.

4. The method of claim 1 wherein the expression of TNF-α is decreased in the subject.

5. The method of claim 1 wherein the expression of IL-6 is decreased in the subject.

6. The method of claim 1 wherein the expression of KC is decreased in the subject.

7. The method of claim 1 wherein the expression of MIP-1α is decreased in the subject.

8. The method of claim 1 wherein the eIF5A1 siRNA targets SEQ ID NO: 53.

9. The method of claim 1 wherein the sense strand of siRNA comprises SEQ ID NO: 54 and the antisense strand comprises SEQ ID NO: 55.

10. The method of claim 1 wherein the expression of IL-1β is decreased in the subject.


SEQUENCE LISTING (117 sequences)

SEQ ID NO: 1 - DNA, 1139 bp, Rattus norvegicus
  CDS (33)..(494)
caggtctagagttggaatcgaagcctcttaaaatggcagatgatttggacttcgagacaggagatgcaggggcctcagccaccttcccaatgcagtgctcagcattacgtaagaatggttttgtggtgctcaagggccggccatgtaagatcgtcgagatgtctacttcgaagactggcaagcatggccatgccaaggtccatctggttggtattgatatttttactgggaagaaatatgaagatatctgcccgtcgactcataacatggatgtccccaacatcaaaaggaatgatttccagctgattggcatccaggatgggtacctatccctgctccaggacagtggggaggtacgagaggaccttcgtctgcctgagggagaccttggcaaggagattgagcagaagtatgactgtggagaagagatcctgatcacagtgctgtccgccatgacagaggaggcagctgttgcaatcaaggccatggcaaaataactggcttccagggtggcggtggtggcagcagtgatccatgagcctacagaggcccctcccccagctctggctgggcccttggctggactcctatccaatttatttgacgttttattttggttttcctcaccccttcaaactgtcggggagaccctgcccttcacctagctcccttggccaggcatgagggagccatggccttggtgaagctacctgcctcttctctcgcagccctgatgggggaaagggagtgggtactgcctgtggtttaggttcccctctccctttttctttttaattcaatttggaatcagaaagctgtggattctggcaaatggtcttgtgtcctttatcccactcaaacccatctggtcccctgttctccatagtccttcacccccaagcaccactgacagactggggaccagcccccttccctgcctgtgtctcttcccaaacccctctataggggtgacaagaagaggagggggggaggggacacgatccctcctcaggcatctgggaaggccttgcccccatgggctttaccctttcctgtgggctttctccctgacacatttgttaaaaatcaaacctgaataaaactacaagtttaatatgaaaaaaaaaaaaaaaaaaaaa

SEQ ID NO: 2 - protein, 154 aa, Rattus norvegicus
MADDLDFETGDAGASATFPMQCSALRKNGFVVLKGRPCKIVEMSTSKTGKHGHAKVHLVGIDIFTGKKYEDICPSTHNMDVPNIKRNDFQLIGIQDGYLSLLQDSGEVREDLRLPEGDLGKEIEQKYDCGEEILITVLSAMTEEAAVAIKAMAK

SEQ ID NO: 3 - DNA, 462 bp, Homo sapiens
atggcagatgacttggacttcgagacaggagatgcaggggcctcagccaccttcccaatgcagtgctcagcattacgtaagaatggctttgtggtgctcaaaggccggccatgtaagatcgtcgagatgtctacttcgaagactggcaagcacggccacgccaaggtccatctggttggtattgacatctttactgggaagaaatatgaagatatctgcccgtcaactcataatatggatgtccccaacatcaaaaggaatgacttccagctgattggcatccaggatgggtacctatcactgctccaggacagcggggaggtacgagaggaccttcgtctccctgagggagaccttggcaaggagattgagcagaagtacgactgtggagaagagatcctgatcacggtgctgtctgccatgacagaggaggcagctgttgcaatcaaggccatggcaaaa

SEQ ID NO: 4 - DNA, 460 bp, Homo sapiens
atggcagacgaaattgatttcactactggagatgccggggcttccagcacttaccctatgcagtgctcggccttgcgcaaaaacggcttcgtggtgctcaaaggacgaccatgcaaaatagtggagatgtcaacttccaaaactggaaagcatggtcatgccaaggttcaccttgttggaattgatattttcacgggcaaaaaatatgaagatatttgtccttctactcacaacatggatgttccaaatattaagagaaatgattatcaactgatatgcattcaagatggttacctttccctgctgacagaaactggtgaagttcgtgaggatcttaaactgccagaaggtgaactaggcaaagaaatagagggaaaatacaatgcaggtgaagatgtacaggtgtctgtcatgtgtgcaatgagtgaagaatatgctgtagccataaaaccctgcaaat

SEQ ID NO: 5 - DNA, 462 bp, Mus musculus
atggcagatgatttggacttcgagacaggagatgcaggggcctcagccaccttcccaatgcagtgctcagcattacgtaagaatggttttgtggtgctcaaaggccggccatgtaagatcgtcgagatgtctacttcgaagactggcaagcatggccatgccaaggtccatctggttggcattgacatttttactgggaagaaatatgaagatatctgcccgtcgactcataatatggatgtccccaacatcaaacggaatgacttccagctgattggcatccaggatgggtacctatccctgctccaggacagtggggaggtacgagaggaccttcgtctgcctgaaggagaccttggcaaggagattgagcagaagtatgactgtggagaagagatcctgatcacagtgctgtctgccatgacagaggaggcagctgttgcaatcaaggccatggcaaaa

SEQ ID NO: 6 - DNA, 606 bp, Rattus norvegicus
  CDS (1)..(453)
gctgtgtattattgggcccataagaaccacatacctgtgctgagtcctgcactcacagacggctcactgggtgacatgatctttttccattcctataaaaacccaggcttggtcctggacatcgttgaagacctgcggctcatcaacatgcaggccattttcgccaagcgcactgggatgatcatcctgggtggaggcgtggtcaagcaccacatcgccaatgctaacctcatgcggaatggagctgactacgctgtttatatcaacacagcccaggagtttgatggctcagactcaggagcccggccagatgaggctgtctcctggggcaagatccggatggatgcacagccagtaaaggtctatgctgatgcatctctggttttccccttgctggtggctgagacattcgcccaaaaggcagatgccttcagagctgagaagaatgaggactgagcagatgggtaaagacggaggcttctgccacacctttatttattatttgcataccaacccctcctgggccctctccttggtcagcagcatcttgagaataaatggcctttttgttggtttctgtaaaaaaaggactttaaaaaaaaaaaa

SEQ ID NO: 7 - protein, 151 aa, Rattus norvegicus
AVYYWAHKNHIPVLSPALTDGSLGDMIFFHSYKNPGLVLDIVEDLRLINMQAIFAKRTGMIILGGGVVKHHIANANLMRNGADYAVYINTAQEFDGSDSGARPDEAVSWGKIRMDAQPVKVYADASLVFPLLVAETFAQKADAFRAEKNED

SEQ ID NO: 8 - DNA, 453 bp, Homo sapiens
tccgtgtattactgggcccagaagaaccacatccctgtgtttagtcccgcacttacagacggctcgctgggcgacatgatcttcttccattcctacaagaacccgggcctggtcctggacatcgttgaggacctgaggctcatcaacacacaggccatctttgccaagtgcactgggatgatcattctgggcgggggcgtggtcaagcaccacattgccaatgccaacctcatgcggaacggggccgactacgctgtttacatcaacacagcccaggagtttgatggctctgactcaggtgcccgaccagacgaggctgtctcctggggcaagatccgggtggatgcacagcccgtcaaggtctatgctgacgcctccctggtcttccccctgcttgtggctgaaacctttgcccagaagatggatgccttcatgcatgagaagaacgaggac

SEQ ID NO: 9 - DNA, 20 bp, Artificial Sequence
  Synthetic primer
tcsaarachggnaagcaygg

SEQ ID NO: 10 - DNA, 42 bp, Artificial Sequence
  Synthetic primer
gcgaagcttccatggctcgagttttttttttttttttttttt

SEQ ID NO: 11 - DNA, 972 bp, Rattus norvegicus
  CDS (1)..(327)
tcgaagaccggtaagcacggccatgccaaggtccatctggttggtattgatatttttactgggaagaaatatgaagatatctgcccgtcgactcataacatggatgtccccaacatcaaaaggaatgatttccagctgattggcatccaggatgggtacctatccctgctccaggacagtggggaggtacgagaggaccttcgtctgcctgagggagaccttggcaaggagattgagcagaagtatgactgtggagaagagatcctgatcacagtgctgtccgccatgacagaggaggcagctgttgcaatcaaggccatggcaaaataactggcttccagggtggcggtggtggcagcagtgatccatgagcctacagaggcccctcccccagctctggctgggcccttggctggactcctatccaatttatttgacgttttattttggttttcctcaccccttcaaactgtcggggagaccctgcccttcacctagctcccttggccaggcatgagggagccatggccttggtgaagctacctgcctcttctctcgcagccctgatgggggaaagggagtgggtactgcctgtggtttaggttcccctctccctttttctttttaattcaatttggaatcagaaagctgtggattctggcaaatggtcttgtgtcctttatcccactcaaacccatctggtcccctgttctccatagtccttcacccccaagcaccactgacagactggggaccagcccccttccctgcctgtgtctcttcccaaacccctctataggggtgacaagaagaggagggggggaggggacacgatccctcctcaggcatctgggaaggccttgcccccatgggctttaccctttcctgtgggctttctccctgacacatttgttaaaaatcaaacctgaataaaactacaagtttaatatgaaaaaaaaaaaaaaaaaaaaa

SEQ ID NO: 12 - protein, 109 aa, Rattus norvegicus
SKTGKHGHAKVHLVGIDIFTGKKYEDICPSTHNMDVPNIKRNDFQLIGIQDGYLSLLQDSGEVREDLRLPEGDLGKEIEQKYDCGEEILITVLSAMTEEAAVAIKAMAK

SEQ ID NO: 13 - DNA, 24 bp, Artificial Sequence
  Synthetic Primer
caggtctagagttggaatcgaagc

SEQ ID NO: 14 - DNA, 30 bp, Artificial Sequence
  Synthetic Primer
atatctcgagccttgattgcaacagctgcc

SEQ ID NO: 15 - DNA, 489 bp, Rattus norvegicus
  CDS (33)..(485)
caggtctagagttggaatcgaagcctcttaaaatggcagatgatttggacttcgagacaggagatgcaggggcctcagccaccttcccaatgcagtgctcagcattacgtaagaatggttttgtggtgctcaagggccggccatgtaagatcgtcgagatgtctacttcgaagactggcaagcatggccatgccaaggtccatctggttggtattgatatttttactgggaagaaatatgaagatatctgcccgtcgactcataacatggatgtccccaacatcaaaaggaatgatttccagctgattggcatccaggatgggtacctatccctgctccaggacagtggggaggtacgagaggaccttcgtctgcctgagggagaccttggcaaggagattgagcagaagtatgactgtggagaagagatcctgatcacagtgctgtccgccatgacagaggaggcagctgttgcaatcaaggctcgag

SEQ ID NO: 16 - protein, 151 aa, Rattus norvegicus
MADDLDFETGDAGASATFPMQCSALRKNGFVVLKGRPCKIVEMSTSKTGKHGHAKVHLVGIDIFTGKKYEDICPSTHNMDVPNIKRNDFQLIGIQDGYLSLLQDSGEVREDLRLPEGDLGKEIEQKYDCGEEILITVLSAMTEEAAVAIKA

SEQ ID NO: 17 - DNA, 20 bp, Artificial Sequence
  Synthetic Primer
gtctgtgtattattgggccc

SEQ ID NO: 18 - DNA, 42 bp, Artificial Sequence; identical to SEQ ID NO: 10
  Synthetic Primer

SEQ ID NO: 19 - DNA, 1299 bp, Homo sapiens
ggcacgagggcggcggcggcggtagaggcggcggcggcggcggcagcgggctcggaggcagcggttgggctcgcggcgagcggacggggtcgagtcagtgcgttcgcgcgagttggaatcgaagcctcttaaaatggcagatgacttggacttcgagacaggagatgcaggggcctcagccaccttcccaatgcagtgctcagcattacgtaagaatggctttgtggtgctcaaaggccggccatgtaagatcgtcgagatgtctacttcgaagactggcaagcacggccacgccaaggtccatctggttggtattgacatctttactgggaagaaatatgaagatatctgcccgtcaactcataatatggatgtccccaacatcaaaaggaatgacttccagctgattggcatccaggatgggtacctatcactgctccaggacagcggggaggtacgagaggaccttcgtctccctgagggagaccttggcaaggagattgagcagaagtacgactgtggagaagagatcctgatcacggtgctgtctgccatgacagaggaggcagctgttgcaatcaaggccatggcaaaataactggctcccaggatggcggtggtggcagcagtgatcctctgaacctgcagaggccccctccccgagcctggcctggctctggcccggtcctaagctggactcctcctacacaatttatttgacgttttattttggttttccccaccccctcaatctgtcggggagcccctgcccttcacctagctcccttggccaggagcgagcgaagctgtggccttggtgaagctgccctcctcttctcccctcacactacagccctggtgggggagaagggggtgggtgctgcttgtggtttagtcttttttttttttttttttttttttttaaattcaatctggaatcagaaagcggtggattctggcaaatggtccttgtgccctccccactcatccctggtctggtcccctgttgcccatagccctttaccctgagcaccaccccaacagactggggaccagccccctcgcctgcctgtgtctctccccaaacccctttagatggggagggaagaggaggagaggggaggggacctgccccctcctcaggcatctgggagggccctgcccccatgggctttacccttccctgcgggctctctccccgacacatttgttaaaatcaaacctgaataaaactacaagtttaatatgaaaaaaaaaaaaaaaaaaaaaaaaaaaaaaaaaaaaaaaaaaaaaaaaaaa

SEQ ID NO: 20 - DNA, 462 bp, Rattus norvegicus
atggcagatgatttggacttcgagacaggagatgcaggggcctcagccaccttcccaatgcagtgctcagcattacgtaagaatggttttgtggtgctcaagggccggccatgtaagatcgtcgagatgtctacttcgaagactggcaagcatggccatgccaaggtccatctggttggtattgatatttttactgggaagaaatatgaagatatctgcccgtcgactcataacatggatgtccccaacatcaaaaggaatgatttccagctgattggcatccaggatgggtacctatccctgctccaggacagtggggaggtacgagaggaccttcgtctgcctgagggagaccttggcaaggagattgagcagaagtatgactgtggagaagagatcctgatcacagtgctgtccgccatgacagaggaggcagctgttgcaatcaaggccatggcaaaa

SEQ ID NO: 21 - protein, 154 aa, Homo sapiens; identical to SEQ ID NO: 2

SEQ ID NO: 22 - protein, 153 aa, Homo sapiens
MADEIDFTTGDAGASSTYPMQCSALRKNGFVVLKGRPCKIVEMSTSKTGKHGHAKVHLVGIDIFTGKKYEDICPSTHNMDVPNIKRNDYQLICIQDGYLSLLTETGEVREDLKLPEGELGKEIEGKYNAGEDVQVSVMCAMSEEYAVAIKPCK

SEQ ID NO: 23 - protein, 154 aa, Mus musculus; identical to SEQ ID NO: 2

SEQ ID NO: 24 - protein, 153 aa, Homo sapiens
MADEIDFTTGDAGASSTYPMQCSALRKNGFVVLKGRPCKIVEMSTSKTGKHGHAKVHLVGIDIFTGKKYEDICPSTHNMDVPNIKRNDYQLICIQDGCLSLLTETGEVREDLKLPEGELGKEIEGKYNAGEDVQVSVMCAMSEEYAVAIKPCK

SEQ ID NO: 25 - DNA, 20 bp, Artificial Sequence
  Synthetic oligonucleotide
gacttggacttcgagacagg

SEQ ID NO: 26 - DNA, 19 bp, Artificial Sequence
  Synthetic oligonucleotide
gcacggccacgccaaggtc

SEQ ID NO: 27 - DNA, 20 bp, Artificial Sequence
  Synthetic oligonucleotide
ggacagcggggaggtacgag

SEQ ID NO: 28 - protein, 153 aa, Artificial Sequence; identical to SEQ ID NO: 24
  Synthetic consensus sequence

SEQ ID NO: 29 - DNA, 1309 bp, Homo sapiens
ggcacgagggtagaggcggcggcggcggcggcagcgggctcggaggcagcggttgggctcgcggcgagcggacggggtcgagtcagtgcgttcgcgcgagttggaatcgaagcctcttaaaatggcagatgacttggacttcgagacaggagatgcaggggcctcagccaccttcccaatgcagtgctcagcattacgtaagaatggctttgtggtgctcaaaggccggccatgtaagatcgtcgagatgtctacttcgaagactggcaagcacggccacgccaaggtccatctggttggtattgacatctttactgggaagaaatatgaagatatctgcccgtcaactcataatatggatgtccccaacatcaaaaggaatgacttccagctgattggcatccaggatgggtacctatcactgctccaggacagcggggaggtacgagaggaccttcgtctccctgagggagaccttggcaaggagattgagcagaagtacgactgtggagaagagatcctgatcacggtgctgtctgccatgacagaggaggcagctgttgcaatcaaggccatggcaaaataactggctcccaggatggcggtggtggcagcagtgatcctctgaacctgcagaggccccctccccgagcctggcctggctctggcccggtcctaagctggactcctcctacacaatttatttgacgttttattttggttttccccaccccctcaatctgtcggggagcccctgcccttcacctagctcccttggccaggagcgagcgaagctgtggccttggtgaagctgccctcctcttctcccctcacactacagccctggtgggggagaagggggtgggtgctgcttgtggtttagtctttttttttttttttttttttttttaaattcaatctggaatcagaaagcggtggattctggcaaatggtccttgtgccctccccactcatccctggtctggtcccctgttgcccatagccctttaccctgagcaccaccccaacagactggggaccagccccctcgcctgcctgtgtctctccccaaacccctttagatggggagggaagaggaggagaggggaggggacctgccccctcctcaggcatctgggagggccctgcccccatgggctttacccttccctgcgggctctctccccgacacatttgttaaaatcaaacctgaataaaactacaagtttaatatgaaaaaaaaaaaaaaaaaaaaaaaaaaaaaaaaaaaaaaaaaaaaaaaaaaaaaaaaaaaaaaaaaaaaaaaaaa

SEQ ID NO: 30 - DNA, 23 bp, Homo sapiens
aaaggaatgacttccagctgatt

SEQ ID NO: 31 - DNA, 23 bp, Homo sapiens
aagatcgtcgagatgtctacttc

SEQ ID NO: 32 - DNA, 23 bp, Homo sapiens
aaggtccatctggttggtattga

SEQ ID NO: 33 - DNA, 23 bp, Homo sapiens
aagctggactcctcctacacaat

SEQ ID NO: 34 - DNA, 23 bp, Homo sapiens
aaagtcgaccttcagtaaggatt

SEQ ID NO: 35 - DNA, 20 bp, Homo sapiens
cctgtctcgaagtccaagtc

SEQ ID NO: 36 - DNA, 20 bp, Homo sapiens
gacttggacttcgagacagg

SEQ ID NO: 37 - DNA, 20 bp, Homo sapiens
ggaccttggcgtggccgtgc

SEQ ID NO: 38 - DNA, 20 bp, Homo sapiens
gcacggccacgccaaggtcc

SEQ ID NO: 39 - DNA, 20 bp, Homo sapiens
ctcgtacctccccgctctcc

SEQ ID NO: 40 - DNA, 19 bp, Homo sapiens
ggacagcggggaggtacga

SEQ ID NO: 41 - DNA, 465 bp, Homo sapiens
atggcagatgacttggacttcgagacaggagatgcaggggcctcagccaccttcccaatgcagtgctcagcattacgtaagaatggctttgtggtgctcaaaggccggccatgtaagatcgtcgagatgtctacttcgaagactggcaagcacggccacgccaaggtccatctggttggtattgacatctttactgggaagaaatatgaagatatctgcccgtcaactcataatatggatgtccccaacatcaaaaggaatgacttccagctgattggcatccaggatgggtacctatcactgctccaggacagcggggaggtacgagaggaccttcgtctccctgagggagaccttggcaaggagattgagcagaagtacgactgtggagaagagatcctgatcacggtgctgtctgccatgacagaggaggcagctgttgcaatcaaggccatggcaaaataa

SEQ ID NO: 42 - DNA, 462 bp, Homo sapiens
atggcagacgaaattgatttcactactggagatgccggggcttccagcacttaccctatgcagtgctcggccttgcgcaaaaacggcttcgtggtgctcaaaggacgaccatgcaaaatagtggagatgtcaacttccaaaactggaaagcatggtcatgccaaggttcaccttgttggaattgatattttcacgggcaaaaaatatgaagatatttgtccttctactcacaacatggatgttccaaatattaagagaaatgattatcaactgatatgcattcaagatggttacctttccctgctgacagaaactggtgaagttcgtgaggatcttaaactgccagaaggtgaactaggcaaagaaatagagggaaaatacaatgcaggtgaagatgtacaggtgtctgtcatgtgtgcaatgagtgaagaatatgctgtagccataaaaccctgcaaataa

SEQ ID NO: 43 - protein, 154 aa, Homo sapiens [sequence omitted]

SEQ ID NO: 44 - DNA, 21 bp, Artificial Sequence
  Synthetic oligonucleotide
aaaggaatgacttccagctga

SEQ ID NO: 45 - DNA, 23 bp, Artificial Sequence
  Synthetic oligonucleotide
aaaggaaugacuuccagcugatt

SEQ ID NO: 46 - DNA, 23 bp, Artificial Sequence
  Synthetic oligonucleotide
ucagcuggaagucauuccuuutt

SEQ ID NO: 47 - DNA, 21 bp, Artificial Sequence
  Synthetic oligonucleotide
aagatcgtcgagatgtctact

SEQ ID NO: 48 - DNA, 23 bp, Artificial Sequence
  Synthetic oligonucleotide
aagaucgucgagaugucuacutt

SEQ ID NO: 49 - DNA, 23 bp, Artificial Sequence
  Synthetic oligonucleotide
aguagacaucucgacgaucuutt

SEQ ID NO: 50 - DNA, 21 bp, Artificial Sequence
  Synthetic oligonucleotide
aaggtccatctggttggtatt

SEQ ID NO: 51 - DNA, 23 bp, Artificial Sequence
  Synthetic oligonucleotide
aagguccaucugguugguauutt

SEQ ID NO: 52 - DNA, 23 bp, Artificial Sequence
  Synthetic oligonucleotide
aauaccaaccagauggaccuutt

SEQ ID NO: 53 - DNA, 21 bp, Artificial Sequence
  Synthetic oligonucleotide
aagctggactcctcctacaca

SEQ ID NO: 54 - DNA, 23 bp, Artificial Sequence
  Synthetic oligonucleotide
aagcuggacuccuccuacacatt

SEQ ID NO: 55 - DNA, 23 bp, Artificial Sequence
  Synthetic oligonucleotide
uguguaggaggaguccagcuutt

SEQ ID NO: 56 - DNA, 21 bp, Artificial Sequence
  Synthetic oligonucleotide
aaagtcgaccttcagtaagga

SEQ ID NO: 57 - DNA, 23 bp, Artificial Sequence
  Synthetic oligonucleotide
aaagucgaccuucaguaaggatt

SEQ ID NO: 58 - DNA, 23 bp, Artificial Sequence
  Synthetic oligonucleotide
uccuuacugaaggucgacuuutt

SEQ ID NO: 59 - DNA, 26 bp, Artificial Sequence
  Synthetic primer
gccaagcttaatggcagatgatttgg

SEQ ID NO: 60 - DNA, 25 bp, Artificial Sequence
  Synthetic primer
ctgaattccagttattttgccatgg

SEQ ID NO: 61 - DNA, 27 bp, Artificial Sequence
  Synthetic primer
aatgaattccgccatgacagaggaggc

SEQ ID NO: 62 - DNA, 42 bp, Artificial Sequence; identical to SEQ ID NO: 10
  Synthetic primer

SEQ ID NO: 63 - DNA, 20 bp, Artificial Sequence
  Synthetic oligonucleotide
cctgtctcgaagtccaagtc

SEQ ID NO: 64 - DNA, 20 bp, Artificial Sequence
  Synthetic oligonucleotide
ggaccttggcgtggccgtgc

SEQ ID NO: 65 - DNA, 20 bp, Artificial Sequence
  Synthetic oligonucleotide
ctcgtacctccccgctctcc

SEQ ID NO: 66 - DNA, 20 bp, Artificial Sequence
  Synthetic oligonucleotide
cgtaccggtacggttccagg

SEQ ID NO: 67 - DNA, 20 bp, Artificial Sequence
  Synthetic oligonucleotide
ggaccttggcgtggccgtgc

SEQ ID NO: 68 - protein, 17 aa, Artificial Sequence
  Synthetic peptide
CRLPEGDLGKEIEQKYD

SEQ ID NO: 69 - DNA, 29 bp, Artificial Sequence
  Synthetic oligonucleotide
aaaggaatgacttccagctgacctgtctc

SEQ ID NO: 70 - DNA, 29 bp, Artificial Sequence
  Synthetic oligonucleotide
aatcagctggaagtcattcctcctgtctc

SEQ ID NO: 71 - DNA, 29 bp, Artificial Sequence
  Synthetic oligonucleotide
aagatcgtcgagatgtctactcctgtctc

SEQ ID NO: 72 - DNA, 29 bp, Artificial Sequence
  Synthetic oligonucleotide
aaagtagacatctcgacgatccctgtctc

SEQ ID NO: 73 - DNA, 29 bp, Artificial Sequence
  Synthetic oligonucleotide
aaggtccatctggttggtattcctgtctc

SEQ ID NO: 74 - DNA, 29 bp, Artificial Sequence
  Synthetic oligonucleotide
aaaataccaaccagatggacccctgtctc

SEQ ID NO: 75 - DNA, 29 bp, Artificial Sequence
  Synthetic oligonucleotide
aagctggactcctcctacacacctgtctc

SEQ ID NO: 76 - DNA, 29 bp, Artificial Sequence
  Synthetic oligonucleotide
aatgtgtaggaggagtccagccctgtctc

SEQ ID NO: 77 - DNA, 29 bp, Artificial Sequence
  Synthetic oligonucleotide
aaagtcgaccttcagtaaggacctgtctc

SEQ ID NO: 78 - DNA, 29 bp, Artificial Sequence
  Synthetic oligonucleotide
aatccttactgaaggtcgactcctgtctc

SEQ ID NO: 79 - RNA, 21 nt, Artificial Sequence
  Synthetic oligonucleotide
aagcuggacuccuccuacaca

SEQ ID NO: 80 - RNA, 21 nt, Artificial Sequence
  Synthetic oligonucleotide
aaacacauccuccucaggucg

SEQ ID NO: 81 - DNA, 21 bp, Artificial Sequence
  Synthetic oligonucleotide
aaaggaatgacttccagctga

SEQ ID NO: 82 - DNA, 21 bp, Artificial Sequence
  Synthetic oligonucleotide
aagatcgtcgagatgtctact

SEQ ID NO: 83 - DNA, 21 bp, Artificial Sequence
  Synthetic oligonucleotide
aaggtccatctggttggtatt

SEQ ID NO: 84 - DNA, 21 bp, Artificial Sequence
  Synthetic oligonucleotide
aagctggactcctcctacaca

SEQ ID NO: 85 - DNA, 21 bp, Artificial Sequence
  Synthetic oligonucleotide
aaagtcgaccttcagtaagga

SEQ ID NO: 86 - RNA, 21 nt, Artificial Sequence
  Synthetic siRNA sequence
nngcuggacuccuccuacaca

SEQ ID NO: 87 - DNA, 21 bp, Artificial Sequence
  Synthetic siRNA sequence
gcuggacuccuccuacacatt

SEQ ID NO: 88 - DNA, 21 bp, Artificial Sequence
  Synthetic siRNA sequence
uguguaggaggaguccagctt

SEQ ID NO: 89 - RNA, 21 nt, Artificial Sequence
  Synthetic siRNA sequence
nnacacauccuccucaggucg

SEQ ID NO: 90 - DNA, 21 bp, Artificial Sequence
  Synthetic siRNA sequence
acacauccuccucaggucgtt

SEQ ID NO: 91 - DNA, 21 bp, Artificial Sequence
  Synthetic siRNA sequence
cgaccugaggaggaugugutt

SEQ ID NO: 92 - DNA, 24 bp, Artificial Sequence
  Synthetic siRNA sequence
cggatggcaacatttagaattagt

SEQ ID NO: 93 - DNA, 24 bp, Artificial Sequence
  Synthetic primer
tgattgagactgtaatcaagaacc

SEQ ID NO: 94 - DNA, 22 bp, Artificial Sequence
  Synthetic primer
ctgatgcccccatgttcgtcat

SEQ ID NO: 95 - DNA, 20 bp, Artificial Sequence
  Synthetic primer
ccaccaccctgttgctgtag

SEQ ID NO: 96 - DNA, 23 bp, Artificial Sequence
  Synthetic siRNA sequence
aacggaaugacuuccagcugatt

SEQ ID NO: 97 - DNA, 21 bp, Artificial Sequence
  Synthetic siRNA sequence
cggaaugacuuccagcugatt

SEQ ID NO: 98 - DNA, 21 bp, Artificial Sequence
  Synthetic siRNA sequence
ucagcuggaagucauuccgtt

SEQ ID NO: 99 - RNA, 19 nt, Artificial Sequence
  Synthetic siRNA sequence
ucagcuggaagucauuccg

SEQ ID NO: 100 - DNA, 20 bp, Artificial Sequence
  Synthetic primer
cgagttggaatcgaagcctc

SEQ ID NO: 101 - DNA, 20 bp, Artificial Sequence
  Synthetic primer
ggttcagaggatcactgctg

SEQ ID NO: 102 - DNA, 26 bp, Artificial Sequence
  Synthetic primer
gccaagcttaatggcagatgatttgg

SEQ ID NO: 103 - DNA, 26 bp, Artificial Sequence
  Synthetic primer
cctgaattccagttattttgccatgg

SEQ ID NO: 104 - DNA, 26 bp, Artificial Sequence
  Synthetic primer
gccaagcttaatggcagatgatttgg

SEQ ID NO: 105 - DNA, 26 bp, Artificial Sequence
  Synthetic primer
gccgaattctccctcaggcagagaag

SEQ ID NO: 106 - DNA, 26 bp, Artificial Sequence
  Synthetic primer
gaggaattcgctgttgcaatcaaggc

SEQ ID NO: 107 - DNA, 26 bp, Artificial Sequence
  Synthetic primer
tttaagctttgtgtcggggagagagc

SEQ ID NO: 108 - DNA, 21 bp, Artificial Sequence
  Synthetic siRNA sequence
gcuggacuccuccuacacatt

SEQ ID NO: 109 - DNA, 21 bp, Artificial Sequence
  Synthetic siRNA sequence
uguguaggaggaguccagctt

SEQ ID NO: 110 - DNA, 21 bp, Artificial Sequence
  Synthetic siRNA sequence
acacauccuccucaggucgtt

SEQ ID NO: 111 - DNA, 21 bp, Artificial Sequence
  Synthetic siRNA sequence
cgaccugaggaggaugugutt

SEQ ID NO: 112 - DNA, 21 bp, Artificial Sequence
  Synthetic siRNA sequence
cggaaugacuuccagcugatt

SEQ ID NO: 113 - DNA, 21 bp, Artificial Sequence
  Synthetic siRNA sequence
agucgaccuucaguaaggctt

SEQ ID NO: 114 - DNA, 27 bp, Artificial Sequence
  Synthetic primer
gctccatggcagatgatttggacttcg

SEQ ID NO: 115 - DNA, 28 bp, Artificial Sequence
  Synthetic primer
cgcgctagccagttattttgccatcgcc

SEQ ID NO: 116 - DNA, 29 bp, Artificial Sequence
  Synthetic primer
gctccatggatgtacccatacgacgtccc

SEQ ID NO: 117 - RNA, 19 nt, Artificial Sequence
  Synthetic Oligonucleotide
cggaaugacuuccagcuga